(19) United States
(12) Patent Application Publication
(10) Pub. No.: US 2026/0272869 A1
(43) Pub. Date: Sep. 17, 2026

(54) ORAL COMPOSITION COMPRISING L-THEANINE

(71) Applicant: NICOVENTURES TRADING LIMITED, London (GB)

(72) Inventors: Deborah SMITH, Berkhamsted (GB); Olivia O'SHEA, Southampton (GB); Tora Mitra GANGULI, Southampton (GB)

(21) Appl. No.: 19/167,981

(22) PCT Filed: Mar. 27, 2024
(86) PCT No.: PCT/GB2024/050825

§ 371 Date: Sep. 23, 2025

(30) Foreign Application Priority Data
Mar. 28, 2023 (GB) 2304518.0

(51) Int. Cl. A61K 31/198; A61K 9/00; A61K 9/08; A61K 9/10; A61K 31/375; A61K 31/592; A61K 31/593; A61K 33/04; A61K 36/258; A61K 36/28; A61K 36/53; A61P 25/26

(52) U.S. Cl. CPC A61K 31/198; A61K 9/0095; A61K 9/08; A61K 9/10; A61K 31/375; A61K 31/592; A61K 31/593; A61K 33/04; A61K 36/258; A61K 36/28; A61K 36/53; A61P 25/26

(57) ABSTRACT

The present disclosure relates to an oral product, a process for producing the oral product, as well as to uses of said oral product. The oral product includes L-theanine, ginseng, vitamin C, Echinacea, and optionally vitamin D or selenium.

### FIELD

The present disclosure relates to an oral product, a process for producing the oral product, as well as to uses of said oral product.

### BACKGROUND

The present disclosure relates to products and compositions intended for human use. The products are configured for oral use and deliver substances such as flavours and/or active ingredients during use.

Products that are easy to administer orally and which can provide a beneficial effect on particular mood states of a human or animal have become popular in recent years. For example, confectionary-type products (e.g. gummies or pastilles) containing vitamins or other mood-enhancing actives provide a convenient and pleasant mode of administration for such active ingredients. Other convenient modes of administration are food and beverages, for example energy drinks. Such products may include active ingredients that are delivered to the user in order to cause a biological response in the user that may enhance physical or mental performance of the user.

Beverages containing theanine and caffeine have been described in U.S. Pat. Nos. 5,780,086 and 6,268,009. More recently, EP1819241 describes beverages containing theanine and caffeine in a ratio of from 5:1 to 1:1.5. Other beverages, such as chamomile tea and other herbal teas, are consumed by some users to aid sleep and relaxation. US20050090512 describes methods of treating extreme physical or mental stress using L-theanine.

It would be desirable to provide an oral product configured for oral use which may deliver active ingredients to the consumer in an enjoyable and effective form, such as in the form of a liquid shot.

### BRIEF SUMMARY

The present disclosure generally provides products configured for oral use, the products comprising a combination of active ingredients. The oral products are in a form suitable for oral use, specifically the oral products are in a liquid form (e.g. as a beverage, such as a shot).

In accordance with some embodiments described herein, there is provided a liquid oral product comprising a combination of active ingredients, wherein the combination of active ingredients comprises: (i) L-theanine; (ii) ginseng; (iii) vitamin C; (iv) Echinacea; and optionally (v) vitamin D or selenium.

In accordance with some embodiments described herein, there is provided a process for preparing a liquid oral product as defined herein, the process comprising: (a) combining active ingredients, wherein the active ingredients include: (i) L-theanine; (ii) ginseng; (iii) vitamin C; (iv) Echinacea; and optionally (v) vitamin D or selenium; (b) contacting the active ingredients with water; and (c) mixing the active ingredients with the water to prepare the liquid oral product.

In accordance with some embodiments described herein, there is provided a use of a combination of active ingredients to provide an improvement in physical wellbeing of a human or animal, wherein the combination of active ingredients comprises (i) L-theanine; (ii) ginseng; (iii) vitamin C; (iv) Echinacea; and optionally (v) vitamin D or selenium. Preferably the combination of active ingredients is present in a liquid oral product as defined herein.

Physical wellbeing is the ability to maintain a healthy quality of life that allows the human or animal to get the most out of daily activities without undue fatigue or physical stress. In accordance with some embodiments described herein, there is provided a use of a combination of active ingredients to boost immune response or to support immunity of a human or animal, wherein the combination of active ingredients comprises (i) L-theanine; (ii) ginseng; (iii) vitamin C; (iv) Echinacea; and optionally (v) vitamin D or selenium. Preferably the combination of active ingredients is present in a liquid oral product as defined herein. In accordance with some embodiments described herein, there is provided a use of a combination of active ingredients to support resilience of a human or animal against infection, wherein the combination of active ingredients comprises (i) L-theanine; (ii) ginseng; (iii) vitamin C; (iv) Echinacea; and optionally (v) vitamin D or selenium. Preferably the combination of active ingredients is present in a liquid oral product as defined herein.

Also provided is a use of a combination of active ingredients to reduce sleepiness of a human or animal, wherein the combination of active ingredients comprises (i) L-theanine; (ii) ginseng; (iii) vitamin C; (iv) Echinacea; and optionally (v) vitamin D or selenium, preferably wherein the combination of active ingredients are present in a liquid oral product as defined herein.

In addition to physical wellbeing, it is important for a healthy quality of life that humans or animals are able to manage mental wellbeing. Such management is supported by the oral product of the present invention due to the observed improvement in alertness, and feelings of being in control. Hence in some embodiments there is provided a use of a combination of active ingredients to improve alertness of a human or animal, wherein the combination of active ingredients comprises (i) L-theanine; (ii) ginseng; (iii) vitamin C; (iv) Echinacea; and optionally (v) vitamin D or selenium, preferably wherein the combination of active ingredients are present in a liquid oral product as defined herein. In some embodiments there is provided a use of a combination of active ingredients to improve feelings of being in control of a human or animal, wherein the combination of active ingredients comprises (i) L-theanine; (ii) ginseng; (iii) vitamin C; (iv) Echinacea; and optionally (v) vitamin D or selenium, preferably wherein the combination of active ingredients are present in a liquid oral product as defined herein.

In preferred embodiments, each of the liquid oral products of the above embodiments may be provided as a liquid product, such as a liquid shot (i.e. a liquid having a volume of up to about 100 mL).

These and other features, aspects, and advantages of the disclosure will be apparent from a reading of the following detailed description. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

For ease of reference, these and further aspects of the present invention are now discussed under appropriate section headings. However, the teachings under each section are not necessarily limited to each particular section.

### BRIEF DESCRIPTION OF THE DRAWINGS

Embodiments of the invention will now be described, by way of example only, with reference to accompanying drawings, in which:

FIG. 1 is a graph showing alertness over time as assessed by a visual analogue mood scale (VAMS), compared to placebo as described in Example 8. Asterisks indicate statistically significant differences at the respective time point. A statistically significant difference versus placebo was observed across the whole session.

FIG. 2 is a graph showing feelings of being “in control of challenges” over time as assessed by a visual analogue scale (VAS), compared to placebo as described in Example 8. A statistically significant difference versus placebo was observed across the whole session.

FIG. 3 is a bar chart showing alertness as assessed by a visual analogue mood scale (VAMS), compared to placebo as described in Example 8. The asterisk indicates that a statistically significant difference versus placebo was observed across the whole session.

FIG. 4 is a bar chart showing feelings of being “in control” as assessed by a visual analogue scale (VAS), compared to placebo as described in Example 8. The asterisk indicates that a statistically significant difference versus placebo was observed across the whole session.

FIG. 5 is a bar chart showing sleepiness as assessed by a visual analogue scale (VAS), compared to placebo as described in Example 8. The asterisk indicates that a statistically significant difference versus placebo was observed across the whole session.

### DETAILED DESCRIPTION

It is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

As used in this specification and the claims, the singular forms “a,” “an,” and “the” include plural referents unless the context clearly dictates otherwise. Reference to “dry % by weight” or “dry weight basis” refers to weight on the basis of dry ingredients (i.e., all ingredients except water). Reference to “wet weight” refers to the weight of the product or composition including water. Unless otherwise indicated, reference to “% by weight” (or “% by weight”) of a product or composition reflects the total wet weight of the product or composition (i.e., including water).

In this specification, unless otherwise stated, the term “about” modifying the quantity of an ingredient in the oral product of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the oral product, or to carry out the methods; and the like. The term “about” also encompasses amounts that differ due to different equilibrium conditions for a product or composition resulting from a particular initial mixture. Whether or not modified by the term “about”, the claims include equivalents to the quantities.

### Oral Product

As described herein, there is provided an oral product comprising a combination of active ingredients, wherein the combination comprises: (i) L-theanine; (ii) ginseng; (iii) vitamin C; (iv) Echinacea; and optionally (v) vitamin D or selenium. The oral product is configured for oral use, and thus for insertion into the user's mouth (i.e. oral cavity). In this specification, unless otherwise stated, the term “oral” in connection to a product refers to a liquid product which, in normal use, is suited to be ingested orally by the user. For example, the product may be in the form of a beverage with a volume of 250 mL or greater, or a liquid shot with a volume of from about 1 mL to about 200 mL, preferably about 20 mL to about 100 mL.

The person skilled in the art will understand that the ranges provided herein may be taken alone or combined with one or more other component ranges to provide a preferred aspect of the invention.

### Combination of Active Ingredients

The product as disclosed herein includes active ingredients. The active ingredients may be naturally occurring or synthetically obtained. The combination of active ingredients comprises suitable active ingredients that cause a biological response in a human or animal. In particular, the combination of active ingredients comprises at least L-theanine, ginseng, vitamin C and Echinacea. Each of the active ingredients may be present in an amount suitable to provide the desired biological response in a human animal.

The specific combination of active ingredients included in the liquid oral product has been found by the inventors to provide an improved effect in the physical wellbeing of the consumer as compared with previously known products and as compared with the consumer not ingesting any other product intended for physical wellbeing. The present inventors have found that the specific combination of active ingredients of the present invention may boost immune response, as well as support immunity of the consumer. Immunity or immune response is the body's ability to defend itself against disease-causing organisms. For example, the combination of active ingredients may strengthen the immune system of the consumer such that it is more resilient against infection. It has also been found that the combination of active ingredients described herein may provide a safe product, with desirable pharmacokinetics (Tmax, Cmax, half life), bioavailability and metabolism.

It has been found that the oral product of the present invention is effective for use over the short, medium or long term. Such use can include daily use of the oral product. For example, a single dosage form of the oral product may be consumed once per day by the user, and the oral product may be used for at least 7 days, at least 14 days, at least 21 days, or at least 28 days.

L-theanine is an amino acid analogue that is also referred to as L-γ-glutamylethylamide and N⁵-ethyl-L-glutamine. It is a water-soluble amino acid isolated from green tea (Camellia sinensis) that has anti-inflammatory activity, antioxidative properties and hepatoprotective effect. The inclusion of L-theanine may improve the effects of the oral product on the immune system. For example, the inclusion of L-theanine may help to regulate immune response, prevent disease, and reduce anti-oxidation stress. Studies have shown that L-theanine can enhance innate immune function by regulating the secretion of immune cytokines.

The L-theanine may be present in the liquid oral product in any suitable amount, such as in an amount of at least about 0.01% by weight, at least about 0.05% by weight, or at least about 0.1% by weight of the oral product. In some embodiments, the L-theanine may be present in an amount of no greater than about 15% by weight, no greater than about 10% by weight, or no greater than about 8% by weight of the oral product.

In some embodiments, the L-theanine may be present in an amount of from about 0.01% to about 15% by weight of the oral product, such as in an amount of from about 0.05% to about 10% by weight of the oral product. In preferred embodiments, the L-theanine is present in an amount of from about 0.1% to about 8% by weight of the oral product.

Ginseng is the root of plants in the genus Panax, which are characterised by the presence of unique steroid saponin phytochemicals (ginsenosides) and gintonin. The putative major active components of ginseng comprise more than 100 species specific triterpene saponins or ‘ginsenosides’. Ginseng and ginseng extracts also contain a range of other potentially bioactive components, including alkaloids, phytosterols, sesquiterpenes, and polyphenols. Ginseng finds use as a dietary supplement in energy drinks or herbal teas, and in traditional medicine. In the present invention ginseng may be modulating the immune function of the consumer, in combination with the other actives such as L-theanine noted above.

The Ginseng may include any suitable form of ginseng, such as Panax ginseng (Korean ginseng), Panax notoginseng (China ginseng) and Panax quinquefolius (American ginseng). The Ginseng may also comprise Ashwagandha (Withania somnifera), commonly known as Indian ginseng. The Ginseng may be present in the form of Ginseng extract, chopped ginseng, shredded Ginseng or powdered ginseng. Preferably, the ginseng is included in the form of Ginseng extract or powdered Ginseng extract. The Ginseng may white ginseng, fresh Ginseng or red ginseng. In some embodiments, the ginseng is red ginseng, such as red Ginseng extract or powdered red Ginseng extract. The inclusion of Ginseng has been found to be beneficial in improving the cognitive effects of the composition, and increasing the boost in immune response or immunity in the user. Ginseng is, for example, known as an immune modulator and has been used for maintaining immune homeostasis and enhancing resistance to illness or microbial attacks through effects on the immune system. Ginseng also has anti-inflammatory and antioxidant properties.

The Ginseng may be present in the liquid oral product in any suitable amount, such as in an amount of at least about 0.01% by weight, at least about 0.05% by weight or at least about 0.1% by weight of the oral product. In some embodiments, the ginseng may be present in an amount of no greater than about 5% by weight, no greater than about 2% by weight, or no greater than about 1% by weight of the oral product.

In some embodiments, the ginseng is present in an amount of from about 0.01% to about 5% by weight of the oral product, such as in an amount of from about 0.05% to about 2% by weight of the oral product. In preferred embodiments, the ginseng is present in an amount of from about 0.1% to about 1% by weight of the oral product.

The combination of active ingredients further comprises vitamin C (ascorbic acid). Vitamin C acts as an antioxidant, helping to protect cells from the damage caused by free radicals. It is also used by the body to make collagen, a protein that may help wounds heal. In addition, vitamin C improves the absorption of iron from plant-based foods and helps the immune system work properly to protect the body from disease. The inclusion of vitamin C may reduce tiredness and fatigue.

The vitamin C may be present in the liquid oral product in any suitable amount, such as in an amount of at least about 0.01% by weight, at least about 0.05% by weight, or at least about 0.1% by weight of the oral product. In some embodiments, the vitamin C may be present in an amount of no greater than about 10% by weight, no greater than about 8% by weight, or no greater than about 5% by weight of the oral product.

In some embodiments, the vitamin C may be present in an amount of from about 0.01% to about 10% by weight, such as from about 0.05% to about 8% by weight, preferably in an amount of from about 0.1% to about 5% by weight of the oral product.

The combination of active ingredients further comprises Echinacea. The Echinacea is either Echinacea purpurea, Echinacea angustifolia or Echinacea pallida and the upper parts (e.g. flowers, fruit, leaves) and/or roots of the plant may be utilized. Echinacea plants contain a variety of active compounds including caffeic acid, alkamides, phenolic acids, rosmarinic acid, polyacetylenes, flavonoids, and cichoric acid. Studies have linked Echinacea and its compounds to many health benefits including reduced inflammation, improved immunity and lower blood sugar levels. The Echinacea may be present in the form of an extract, chopped or shredded or powdered. Preferably, the Echinacea is included in the form of an extract.

The Echinacea may be present in the liquid oral product in any suitable amount, such as in an amount of at least about 0.001% by weight, at least about 0.01% by weight or at least about 0.05% by weight of the oral product. In some embodiments, the Echinacea may be present in an amount of no greater than about 20% by weight, no greater than about 15% by weight, or no greater than about 10% by weight of the oral product.

In some embodiments, the Echinacea is present in an amount of from about 0.001% to about 20% by weight of the oral product, such as in an amount of from about 0.01% to about 15% by weight of the oral product. In preferred embodiments, the Echinacea is present in an amount of from about 0.05% to about 10% by weight of the oral product.

Vitamin D is a group of fat-soluble secosteroids responsible for increasing intestinal absorption of calcium, magnesium, and phosphate. In humans the most important compounds in this group are vitamin D₃ (cholecalciferol) and D₂ (ergocalciferol). Vitamin D as used herein thus refers to D₂, D₃ or both. The structural difference between vitamin D₂ and D₃ is in the side chain, which contains a double bond, between carbons 22 and 23, and a methyl group on carbon 24 in vitamin D₂. As well as calcium, magnesium, and phosphate absorption, vitamin D affects the immune system and vitamin D receptors (VDRs) are expressed in several white blood cells, including monocytes and activated T and B cells. In general, vitamin D functions to activate the innate and dampen the adaptive immune systems with antibacterial, antiviral and anti-inflammatory effects.

When present in the liquid oral product, the vitamin D may be used in any suitable amount, such as in an amount of at least about 0.0000001% by weight, at least about 0.0000025% by weight or at least about 0.00001% by weight of the oral product. In some embodiments, the vitamin D may be present in an amount of no greater than about 0.1% by weight, no greater than about 0.01% by weight, no greater than about 0.005% by weight, or no greater than about 0.001% by weight of the oral product.

In some embodiments, the vitamin D is present in an amount of from about 0.000001% to about 0.1% by weight of the oral product, such as in an amount of from about 0.0000025% to about 0.005% by weight of the oral product. In preferred embodiments, the vitamin D is present in an amount of from about 0.00001% to about 0.001% by weight of the oral product.

Selenium is a trace element that is nutritionally essential for humans as a component of multiple selenoproteins that play critical roles in reproduction, thyroid hormone metabolism, DNA synthesis, and protection from oxidative damage and infection. When present in the liquid oral product, the selenium may be used in any suitable amount such as in an amount of at least 0.0000001% by weight, at least about 0.0000005% by weight, or at least about 0.000001% by weight of the oral product. In some embodiments, the selenium may be present in an amount of no greater than about 0.001% by weight, no greater than about 0.0005% by weight, or no greater than about 0.0001% by weight. In some embodiments, the selenium may be provided in the form of one or more inorganic forms (e.g. selenate and selenite salts), one or more organic forms (e.g. selenomethionine and selenocysteine) and/or mixtures thereof.

In some embodiments, selenium is present in an amount of from about 0.0000001% by weight to about 0.001% by weight of the oral product, such as in an amount of from about 0.0000005% by weight to about 0.0005% by weight of the oral product. In preferred embodiments, the selenium is present in an amount of from about 0.000001% by weight to about 0.0001% by weight of the oral product.

In addition to L-theanine, ginseng, vitamin C, Echinacea and optionally vitamin D or selenium, the combination of active ingredients may include one or more additional active ingredients. The additional active ingredients may be any suitable active ingredient that aids immune response, immunity or resilience against infection, and may for example be selected from nutraceuticals, nootropics, psycho-actives. The additional active ingredient may be naturally occurring or synthetically obtained.

Non-limiting examples of additional active ingredients include those falling in the categories of botanical ingredients, amino acids, nicotine components, and/or nutraceutical, and medicinal ingredients (e.g., vitamins, such as A, B1, B2, B3, B5, B6, B7, B9, B12, and/or cannabinoids, such as tetrahydrocannabinol (THC) and cannabidiol (CBD)).

The additional active ingredient may comprise for example taurine, theine, vitamins such as B vitamins (e.g. B1, B2, B3, B5, B6, B7, B9 and/or B12), vitamin A, vitamin E, or vitamin K, melatonin, gamma-aminobutyric acid (GABA), cannabinoids, or constituents, derivatives, or combinations thereof.

In some embodiments, the one or more additional active ingredients is selected from a botanical ingredient (e.g., lavender, peppermint, chamomile, basil, rosemary, ginger, maca, lemon balm, and tisanes), an amino acid (e.g., taurine, phenylalanine, tyrosine, GABA, and tryptophan), a cannabinoid, and/or a nutraceutical, or medicinal ingredient (e.g., a vitamin, such as a B vitamin).

In some embodiments, the combination of active ingredients further comprises lemon balm. Lemon balm is also referred to as Melissa officinalis and is a perennial herbaceous plant in the mint family. Lemon balm contains a complex mixture of terpenes, terpenoids, flavonoids, polyphenols, and other molecules. The lemon balm may be in the form of leaves, stems or roots from the lemon balm plant. In some embodiments, the lemon balm is included in the form of lemon balm leaves, such as chopped, shredded or powdered lemon balm leaves. In some embodiments, the lemon balm is included in the form of a lemon balm extract, such as an aqueous extract of lemon balm. In some embodiments, the lemon balm is included in the form of lemon balm essential oil. Lemon balm has previously been found to exert antioxidant, anti-inflammatory and neuroprotective properties.

The lemon balm may be present in any suitable amount, such as in an amount of at least about 0.01% by weight, at least 0.05% by weight or at least 0.1% by weight of the oral product. In some embodiments, the lemon balm is present in an amount of no greater than about 3% by weight or no greater than about 2% by weight of the oral product.

In some embodiments, the lemon balm is present in an amount of from about 0.01% to about 3% by weight of the oral product, such as in an amount of from about 0.05% by weight to about 3% by weight. In preferred embodiments, the lemon balm is present in an amount of from about 0.1% to about 2% by weight of the oral product.

In some embodiments, the combination of active ingredients further comprises chamomile. Chamomile (Matricaria recuita) is a flowering plant in the daisy (Asteraceae) family. Native to Europe and Western Asia, it is now found around the world. There are two different chamomile plants, German chamomile and Roman chamomile. German chamomile is generally considered the more potent variety and the type most widely used for medicinal purposes. Different classes of bioactive constituents are present in chamomile, such as flavanols (apigenin, luteolin, quercetin) coumarins and terpenoids. Research has shown chamomile to have benefits when it comes to boosting immunity or the immune system to help fight infections. Chamomile is also known to have anti-inflammatory and anti-oxidant effects.

The chamomile may be in the form of chamomile extract. The chamomile (e.g. chamomile extract) may be present in an amount of at least about 0.001% by weight, at least about 0.005% by weight or at least about 0.01% by weight of the oral product. In some embodiments, the chamomile (e.g. chamomile extract) is present in an amount of no greater than about 1% by weight, no greater than about 0.5% by weight, or no greater than about 0.1% by weight of the oral product.

In some preferred embodiments, the combination of active ingredients comprises L-theanine, ginseng, vitamin C, Echinacea, and vitamin D. In other preferred embodiments, the combination of active ingredients comprises L-theanine, ginseng, vitamin C, Echinacea, and selenium.

In some embodiments, the combination of active ingredients comprises L-theanine, ginseng, vitamin C, Echinacea, and lemon balm. In some embodiments, the combination of active ingredients comprises L-theanine, ginseng, vitamin C, Echinacea, and chamomile extract. In some embodiments, the combination of active ingredients comprises L-theanine, ginseng, vitamin C, Echinacea, lemon balm, and chamomile extract.

In some embodiments, the combination of active ingredients comprises L-theanine, ginseng, vitamin C, Echinacea, vitamin D, and lemon balm. In some embodiments, the combination of active ingredients comprises L-theanine, ginseng, vitamin C, Echinacea, vitamin D, and chamomile extract. In some embodiments, the combination of active ingredients comprises L-theanine, ginseng, vitamin C, Echinacea, vitamin D, lemon balm, and chamomile extract.

In some embodiments, the combination of active ingredients comprises L-theanine, ginseng, vitamin C, Echinacea, selenium, and lemon balm. In some embodiments, the combination of active ingredients comprises L-theanine, ginseng, vitamin C, Echinacea, selenium, and chamomile extract. In some embodiments, the combination of active ingredients comprises L-theanine, ginseng, vitamin C, Echinacea, selenium, lemon balm, and chamomile extract.

In any of these embodiments, the term “comprises” may be replaced with the term “consists essentially of” or “consists of”. In other words, in some embodiments the combination of active ingredients may be limited to L-theanine, ginseng, vitamin C, Echinacea, vitamin D, lemon balm, and chamomile extract. In other embodiments, the combination of active ingredients may be limited to L-theanine, ginseng, vitamin C, Echinacea, selenium, lemon balm, and chamomile extract

In some embodiments, the combination of active ingredients comprises (i) from about 0.01% to about 15% L-theanine by weight of the oral product, (ii) from about 0.01% to about 5% ginseng by weight of the oral product, (iii) from about 0.01% to about 10% vitamin C by weight of the oral product, and (iv) from about 0.001% to about 20% Echinacea by weight of the oral product.

In some embodiments, the combination of active ingredients comprises (i) from about 0.05% to about 10% L-theanine by weight of the oral product, (ii) from about 0.01% to about 5% ginseng by weight of the oral product, (iii) from about 0.01% to about 10% vitamin C by weight of the oral product, and (iv) from about 0.001% to about 20% Echinacea by weight of the oral product.

In some embodiments, the combination of active ingredients comprises (i) from about 0.1% to about 8% L-theanine by weight of the oral product, (ii) from about 0.01% to about 5% ginseng by weight of the oral product, (iii) from about 0.01% to about 10% vitamin C by weight of the oral product, and (iv) from about 0.001% to about 20% Echinacea by weight of the oral product.

In some embodiments, the combination of active ingredients comprises (i) from about 0.01% to about 15% L-theanine by weight of the oral product, (ii) from about 0.05% to about 2% ginseng by weight of the oral product, (iii) from about 0.01% to about 10% vitamin C by weight of the oral product, and (iv) from about 0.001% to about 20% Echinacea by weight of the oral product.

In some embodiments, the combination of active ingredients comprises (i) from about 0.01% to about 15% L-theanine by weight of the oral product, (ii) from about 0.1% to about 1% ginseng by weight of the oral product, (iii) from about 0.01% to about 10% vitamin C by weight of the oral product, and (iv) from about 0.001% to about 20% Echinacea by weight of the oral product.

In some embodiments, the combination of active ingredients comprises (i) from about 0.01% to about 15% L-theanine by weight of the oral product, (ii) from about 0.01% to about 5% ginseng by weight of the oral product, (iii) from about 0.05% to about 8% vitamin C by weight of the oral product, and (iv) from about 0.001% to about 20% Echinacea by weight of the oral product.

In some embodiments, the combination of active ingredients comprises (i) from about 0.01% to about 15% L-theanine by weight of the oral product, (ii) from about 0.01% to about 5% ginseng by weight of the oral product, (iii) from about 0.1% to about 5% vitamin C by weight of the oral product, and (iv) from about 0.001% to about 20% Echinacea by weight of the oral product.

In some embodiments, the combination of active ingredients comprises (i) from about 0.01% to about 15% L-theanine by weight of the oral product, (ii) from about 0.01% to about 5% ginseng by weight of the oral product, (iii) from about 0.01% to about 10% vitamin C by weight of the oral product, and (iv) from about 0.01% to about 15% Echinacea by weight of the oral product.

In some embodiments, the combination of active ingredients comprises (i) from about 0.01% to about 15% L-theanine by weight of the oral product, (ii) from about 0.01% to about 5% ginseng by weight of the oral product, (iii) from about 0.01% to about 10% vitamin C by weight of the oral product, and (iv) from about 0.05% to about 10% Echinacea by weight of the oral product.

In some embodiments, the combination of active ingredients comprises (i) from about 0.05% to about 10% L-theanine by weight of the oral product, (ii) from about 0.05% to about 2% ginseng by weight of the oral product, (iii) from about 0.01% to about 10% vitamin C by weight of the oral product, and (iv) from about 0.001% to about 20% Echinacea by weight of the oral product.

In some embodiments, the combination of active ingredients comprises (i) from about 0.05% to about 10% L-theanine by weight of the oral product, (ii) from about 0.05% to about 2% ginseng by weight of the oral product, (iii) from about 0.05% to about 8% vitamin C by weight of the oral product, and (iv) from about 0.001% to about 20% Echinacea by weight of the oral product.

In some embodiments, the combination of active ingredients comprises (i) from about 0.05% to about 10% L-theanine by weight of the oral product, (ii) from about 0.05% to about 2% ginseng by weight of the oral product, (iii) from about 0.01% to about 10% vitamin C by weight of the oral product, and (iv) from about 0.01% to about 15% Echinacea by weight of the oral product.

In some embodiments, the combination of active ingredients comprises (i) from about 0.05% to about 10% L-theanine by weight of the oral product, (ii) from about 0.05% to about 2% ginseng by weight of the oral product, (iii) from about 0.05% to about 8% vitamin C by weight of the oral product, and (iv) from about 0.01% to about 15% Echinacea by weight of the oral product.

In some embodiments, the combination of active ingredients comprises (i) from about 0.1% to about 8% L-theanine by weight of the oral product, (ii) from about 0.05% to about 2% ginseng by weight of the oral product, (iii) from about 0.05% to about 8% vitamin C by weight of the oral product, and (iv) from about 0.01% to about 15% Echinacea by weight of the oral product.

In some embodiments, the combination of active ingredients comprises (i) from about 0.1% to about 8% L-theanine by weight of the oral product, (ii) from about 0.1% to about 1% ginseng by weight of the oral product, (iii) from about 0.05% to about 8% vitamin C by weight of the oral product, and (iv) from about 0.01% to about 15% Echinacea by weight of the oral product.

In some embodiments, the combination of active ingredients comprises (i) from about 0.1% to about 8% L-theanine by weight of the oral product, (ii) from about 0.1% to about 1% ginseng by weight of the oral product, (iii) from about 0.1% to about 5% vitamin C by weight of the oral product, and (iv) from about 0.01% to about 15% Echinacea by weight of the oral product.

In some embodiments, the combination of active ingredients comprises (i) from about 0.1% to about 8% L-theanine by weight of the oral product, (ii) from about 0.1% to about 1% ginseng by weight of the oral product, (iii) from about 0.1% to about 5% vitamin C by weight of the oral product, and (iv) from about 0.05% to about 10% Echinacea by weight of the oral product.

In any of these embodiments, the combination of active ingredients may further comprise vitamin D in an amount of from about 0.000001% to about 0.1% by weight of the oral product.

In any of these embodiments, the combination of active ingredients may further comprise vitamin D in an amount of from about 0.000001% to about 0.01% by weight of the oral product.

In any of these embodiments, the combination of active ingredients may further comprise vitamin D in an amount of from about 0.0000025% to about 0.005% by weight of the oral product.

In any of these embodiments, the combination of active ingredients may further comprise vitamin D in an amount of from about 0.00001 to about 0.001% by weight of the oral product.

In any of these embodiments, the combination of active ingredients may further comprise selenium in an amount of from about 0.000001% to about 0.001% by weight of the oral product. Selenium may be used as an alternative or in addition to vitamin D. Preferably selenium is used as an alternative to vitamin D such that the oral product is free of vitamin D.

In any of these embodiments, the combination of active ingredients may further comprise selenium in an amount of from about 0.000005% to about 0.0005% by weight of the oral product.

In any of these embodiments, the combination of active ingredients may further comprise selenium in an amount of from about 0.00001% to about 0.0001% by weight of the oral product.

For example, in some embodiments, the combination of active ingredients comprises (i) from about 0.01% to about 15% L-theanine by weight of the oral product, (ii) from about 0.01% to about 5% ginseng by weight of the oral product, (iii) from about 0.01% to about 10% vitamin C by weight of the oral product, (iv) from about 0.001% to about 20% Echinacea by weight of the oral product, and (v) vitamin D in an amount of from about 0.000001% to about 0.1% by weight of the oral product. In some embodiments, the combination of active ingredients comprises (i) from about 0.01% to about 15% L-theanine by weight of the oral product, (ii) from about 0.01% to about 5% ginseng by weight of the oral product, (iii) from about 0.01% to about 10% vitamin C by weight of the oral product, (iv) from about 0.001% to about 20% Echinacea by weight of the oral product, and (v) vitamin D in an amount of from about 0.000001% to about 0.01% by weight of the oral product. In some embodiments, the combination of active ingredients comprises (i) from about 0.05% to about 10% L-theanine by weight of the oral product, (ii) from about 0.05% to about 2% ginseng by weight of the oral product, (iii) from about 0.05% to about 8% vitamin C by weight of the oral product, (iv) from about 0.01% to about 15% Echinacea by weight of the oral product, and (v) vitamin D in an amount of from about 0.0000025% to about 0.005% by weight of the oral product. In some embodiments, the combination of active ingredients comprises (i) from about 0.1% to about 8% L-theanine by weight of the oral product, (ii) from about 0.1% to about 1% ginseng by weight of the oral product, (iii) from about 0.1% to about 5% vitamin C by weight of the oral product, (iv) from about 0.05% to about 10% Echinacea by weight of the oral product, and (v) vitamin D in an amount of from about 0.00001 to about 0.001% by weight of the oral product.

In some embodiments, the combination of active ingredients comprises (i) from about 0.01% to about 15% L-theanine by weight of the oral product, (ii) from about 0.01% to about 5% ginseng by weight of the oral product, (iii) from about 0.01% to about 10% vitamin C by weight of the oral product, (iv) from about 0.001% to about 20% Echinacea by weight of the oral product, and (v) selenium in an amount of from about 0.000001% to about 0.01% by weight of the oral product. In some embodiments, the combination of active ingredients comprises (i) from about 0.05% to about 10% L-theanine by weight of the oral product, (ii) from about 0.05% to about 2% ginseng by weight of the oral product, (iii) from about 0.05% to about 8% vitamin C by weight of the oral product, (iv) from about 0.01% to about 15% Echinacea by weight of the oral product, and (v) selenium in an amount of from about 0.000005% to about 0.0005% by weight of the oral product. In some embodiments, the combination of active ingredients comprises (i) from about 0.1% to about 8% L-theanine by weight of the oral product, (ii) from about 0.1% to about 1% ginseng by weight of the oral product, (iii) from about 0.1% to about 5% vitamin C by weight of the oral product, (iv) from about 0.05% to about 10% Echinacea by weight of the oral product, and (v) selenium in an amount of from about 0.00001% to about 0.0001% by weight of the oral product.

In any of these embodiments, lemon balm may be present in an amount of from about 0.01% to about 3% by weight of the oral product. The lemon balm may be present in an amount of about 0.05% to about 3% by weight of the oral product. In preferred embodiments, lemon balm may be present in an amount of from about 0.1% to about 2% by weight of the oral product. Alternatively or in addition, chamomile may be present in an amount of from about 0.001% to about 0.1% by weight of the oral product. Chamomile may be present in an amount of from about 0.005% to about 0.5% by weight of the oral product. In preferred embodiments, chamomile may be present in an amount of from about 0.01% to about 0.1% by weight of the oral product.

For example, in some embodiments, the combination of active ingredients comprises (i) from about 0.01% to about 15% L-theanine by weight of the oral product, (ii) from about 0.01% to about 5% ginseng by weight of the oral product, (iii) from about 0.01% to about 10% vitamin C by weight of the oral product, (iv) from about 0.001% to about 20% Echinacea by weight of the oral product, (v) vitamin D in an amount of from about 0.000001% to about 0.1% by weight of the oral product, and (vi) lemon balm in an amount of from about 0.01% to about 3% by weight of the oral product and/or (vii) chamomile in an amount of from about 0.001% to about 0.1% by weight of the oral product. In some embodiments, the combination of active ingredients comprises (i) from about 0.01% to about 15% L-theanine by weight of the oral product, (ii) from about 0.01% to about 5% ginseng by weight of the oral product, (iii) from about 0.01% to about 10% vitamin C by weight of the oral product, (iv) from about 0.001% to about 20% Echinacea by weight of the oral product, (v) vitamin D in an amount of from about 0.000001% to about 0.01% by weight of the oral product, and (vi) lemon balm in an amount of from about 0.01% to about 3% by weight of the oral product and/or (vii) chamomile in an amount of from about 0.001% to about 0.1% by weight of the oral product. In some embodiments, the combination of active ingredients comprises (i) from about 0.05% to about 10% L-theanine by weight of the oral product, (ii) from about 0.05% to about 2% ginseng by weight of the oral product, (iii) from about 0.05% to about 8% vitamin C by weight of the oral product, (iv) from about 0.01% to about 15% Echinacea by weight of the oral product, (v) vitamin D in an amount of from about 0.0000025% to about 0.005% by weight of the oral product, and (vi) lemon balm in an amount of from about 0.05% to about 3% by weight of the oral product and/or (vii) chamomile in an amount of from about 0.005% to about 0.5% by weight of the oral product. In some embodiments, the combination of active ingredients comprises (i) from about 0.1% to about 8% L-theanine by weight of the oral product, (ii) from about 0.1% to about 1% ginseng by weight of the oral product, (iii) from about 0.1% to about 5% vitamin C by weight of the oral product, (iv) from about 0.05% to about 10% Echinacea by weight of the oral product, (v) vitamin D in an amount of from about 0.00001 to about 0.001% by weight of the oral product, and (vi) lemon balm in an amount of from about 0.1% to about 2% by weight of the oral product and/or (vii) chamomile in an amount of from about 0.01% to about 0.1% by weight of the oral product.

For example, in some embodiments, the combination of active ingredients comprises (i) from about 0.01% to about 15% L-theanine by weight of the oral product, (ii) from about 0.01% to about 5% ginseng by weight of the oral product, (iii) from about 0.01% to about 10% vitamin C by weight of the oral product, (iv) from about 0.001% to about 20% Echinacea by weight of the oral product, (v) selenium in an amount of from about 0.000001% to about 0.001% by weight of the oral product, and (vi) lemon balm in an amount of from about 0.01% to about 3% by weight of the oral product and/or (vii) chamomile in an amount of from about 0.001% to about 0.1% by weight of the oral product. In some embodiments, the combination of active ingredients comprises (i) from about 0.05% to about 10% L-theanine by weight of the oral product, (ii) from about 0.05% to about 2% ginseng by weight of the oral product, (iii) from about 0.05% to about 8% vitamin C by weight of the oral product, (iv) from about 0.01% to about 15% Echinacea by weight of the oral product, (v) selenium in an amount of from about 0.000005% to about 0.0005% by weight of the oral product, and (vi) lemon balm in an amount of from about 0.05% to about 3% by weight of the oral product and/or (vii) chamomile in an amount of from about 0.005% to about 0.5% by weight of the oral product. In some embodiments, the combination of active ingredients comprises (i) from about 0.1% to about 8% L-theanine by weight of the oral product, (ii) from about 0.1% to about 1% ginseng by weight of the oral product, (iii) from about 0.1% to about 5% vitamin C by weight of the oral product, (iv) from about 0.05% to about 10% Echinacea by weight of the oral product, (v) selenium in an amount of from about 0.00001% to about 0.0001% by weight of the oral product, and (vi) lemon balm in an amount of from about 0.1% to about 2% by weight of the oral product and/or (vii) chamomile in an amount of from about 0.01% to about 0.1% by weight of the oral product.

The amounts of the active ingredients as described above have been found by the inventors to provide a beneficial effect of boosting immunity and improving resilience against infection for the user, while also providing a safe product. The amounts may be tailored such that the product has high efficacy while also ensuring consumer safety and avoiding any overdose of actives.

Other suitable additional active ingredients are described below.

In some embodiments, the combination of active ingredients further comprises a botanical active ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include Bacopa monniera, baobab, basil, Centella asiatica, Chai-hu, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glaucal, ginger, Ginkgo biloba, Griffonia simplicifolia, guarana, cannabis, hemp, hops, jasmine, kava, lavender, lemongrass, liquorice, lutein, maca, matcha, Nardostachys chinensis, oil-based extract of Viola odorata, peppermint, piperine, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

The combination of active ingredients may comprise a B vitamin (e.g. vitamin B1, vitamin B2, vitamin B3, vitamin B5, vitamin B6, vitamin B7, vitamin B9, vitamin B12; preferably one or more of vitamin B6 and vitamin B12). Where present, the B vitamin (such as vitamin B6 and/or vitamin B12) may be present in an amount of from about 0.001% to about 5% by weight of the oral product, preferably from about 0.01% to about 2.5% by weight of the oral product.

The combination of active ingredients may further comprise a vitamin selected from vitamin A, vitamin E and vitamin K, or mixtures thereof.

The combination of active ingredients may further comprise an amino acid, such as an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, gamma-aminobutyric acid (GABA), taurine (2-aminoethanesulfonic acid), hydroxyproline, and beta-alanine. In some embodiments, the combination of active ingredients comprises GABA.

In some embodiments, the combination of active ingredients comprises a cannabinoid. The cannabinoid may be a derivative or extract of cannabis. Cannabinoids are a class of natural or synthetic chemical compounds which act on cannabinoid receptors (i.e., CB1 and CB2) in cells that repress neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids. In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabimolic acid (THCA), tetrahydrocannabivarinic acid (THCV A), and mixtures thereof. In some embodiments, the cannabinoid is or comprises at least tetrahydrocannabinol (THC). In some embodiments, the cannabinoid is or comprises at least cannabidiol (CBD).

In some embodiments, the combination of active ingredients comprises a cannabinoid (such as cannabidiol) in an amount of at least about 0.001% by weight of the oral product, such as in a range from about 0.001% to about 10% by weight, such as from about 0.01% to about 5% by weight, such as from about 0.1% to about 2.5% by weight, such as from 0.5% to about 1% by weight of the oral product.

In some embodiments, the combination of active ingredients comprises magnesium glycinate. Where present, magnesium glycinate may be included in an amount of from about 0.1% to about 10% by weight, such as from about 0.5% to about 5% by weight, or from about 0.5% to about 1% by weight of the oral product.

In some embodiments, the combination of active ingredients further comprises piperine. Piperine is an alkaloid found in fruits of the pepper species in the family Piperaceae, such as black pepper (Piper nigrum), long pepper (Piper longum), red long pepper (Piper retrofactum) and voatsiperifery pepper (Piper borbonense). Piperine may be beneficial to include in the oral product because of its ability to enhance the bioavailability of one or more compounds included in the oral product. The origin of piperine is not limited; in some embodiments it may be obtained from a black pepper extract.

Piperine has, for example, been found to enhance the bioavailability of one or more P-glycoprotein substrate compounds obtained from saffron or Ashwagandha. Specifically, piperine has been found to enhance the bioavailability of crocetin, crocin, withanolide A, and withanone. Each of these compounds are P-glycoprotein substrates and their permeation across the blood-brain barrier has been found to be enhanced by combining said compounds with piperine. This is unexpected because the enhancement was not observed with another P-gp inhibitor (quercetin), and/or with other P-glycoprotein substrate compounds, e.g. ursolic acid obtained from Holy Basil. In some embodiments the combination of active ingredients may therefore include ginseng in the form of Ashwagandha and further include piperine. In such embodiments, piperine may enhance/improve the bioavailability of withanolide A and/or withanone provided by the Ashwagandha in the oral product. The enhancement/improvement is relative to an oral product without piperine, and refers to the blood-brain barrier permeation of the P-glycoprotein substrates withanolide A and/or withanone.

In some embodiments the combination of active ingredients may alternatively or additionally include crocin and/or crocetin. Crocin and/or crocetin may be obtained from a saffron extract. Saffron is disclosed herein as a possible flavouring agent for the oral product. In a similar manner to above, piperine may enhance/improve the bioavailability of crocin and/or crocetin in the oral product relative to an oral product without piperine. The enhancement/improvement refers to the blood-brain barrier permeation of the P-glycoprotein substrates crocin and/or crocetin.

The benefit of such enhancement of the blood-brain barrier permeation is that it may allow lower amounts of the active ingredients to be included in the oral product, thereby mitigating or preventing any issues that may arise from higher amounts, e.g. issues of solubility, sensorials, compliance, safety etc., The inclusion of lower amounts of actives is especially beneficial when the oral product is prepared as a liquid in the form of a shot, i.e. a drink that may be consumed quickly. The liquid oral product may, for example, have a volume of from about 10 mL to about 100 mL, such as from about 25 mL to about 75 mL. In such embodiments, the liquid oral product may be considered to be a shot. For example, the volume of liquid may be about 60 mL or about 30 mL. Inclusion of lower amounts should not, however, compromise the effect of the oral product and piperine may therefore be used for this purpose.

In some embodiments, the oral product may comprise about 0.0001 wt % to about 0.1 wt % of piperine, based on the total weight of the oral product. In preferred embodiments the oral product may comprise about 0.0005 wt % to about 0.05 wt % of piperine, based on the total weight of the oral product.

For the avoidance of doubt, combinations of the above end points are explicitly envisaged by the present disclosure. This applies to any of the ranges disclosed herein.

### Ratios of Active Ingredients

The ratios of the active ingredients in the oral product may be selected to increase the effects of the oral product on the consumer.

In some embodiments, the L-theanine and ginseng are present in a weight ratio of from about 1:10 to about 35:1, such as from about 1:5 to about 30:1. In preferred embodiments, the L-theanine and ginseng are present in a weight ratio of from about 1:1 to about 25:1.

It has been found by the present inventors that the weight ratios involving vitamin C may be beneficial for the effects of the oral product on the consumer. In some embodiments, the vitamin C and L-theanine are present in a weight ratio of from about 1:10 to about 10:1, such as from about 1:10 to about 8:1. In preferred embodiments, the vitamin C and L-theanine are present in a weight ratio of from about 1:10 to about 5:1.

In some embodiments, the vitamin C and ginseng are present in a weight ratio of from about 1:10 to about 10:1, such as about 1:5 to about 10:1. In preferred embodiments, the vitamin C and ginseng are present in a weight ratio of from about 1:5 to about 5:1.

The amount of Echinacea included in the product may have a desirable effect on boosting immunity and supporting resilience against infection. In some embodiments, the Echinacea and L-theanine are present in a weight ratio of from about 1:10 to about 25:1, such as a weight ratio of from about 1:10 to about 20:1. In preferred embodiments, the Echinacea and L-theanine are present in a weight ratio of from about 1:5 to about 15:1.

In some embodiments, the ginseng and Echinacea are present in a weight ratio of from about 1:25 to about 5:1, such as from about 1:25 to about 4:1. In preferred embodiments, the ginseng and Echinacea are present in a weight ratio of from about 1:10 to about 4:1.

In any of the above embodiments, the combination of active ingredients may further comprise vitamin D. When vitamin D is present, the vitamin C and vitamin D may be present in a weight ratio of about 120000:1 to about 1000:1, such as from about 120000:1 to 2000:1. In preferred embodiments, the vitamin C and vitamin D may be present in a weight ratio of from about 7000:1 to about 2500:1.

In any of the above embodiments, the combination of active ingredients may further comprise selenium. When selenium is present, the vitamin C and selenium may be present in a weight ratio of about 10000000:1 to about 10:1. In preferred embodiments, the vitamin C and selenium may be present in a weight ratio of from about 100000:1 to about 100:1. Selenium may be present as an alternative or in addition to vitamin D. In preferred embodiments selenium is included as an alternative to vitamin D. Hence, the oral product may be defined by a vitamin C and selenium weight ratio, rather than a vitamin C and vitamin D weight ratio.

As already explained above, the combination of active ingredients may further comprise lemon balm and/or chamomile. In some embodiments, the L-theanine and lemon balm may be present in a weight ratio of from about 1:10 to about 35:1, such as from about 1:5 to about 30:1. In preferred embodiments, the L-theanine and lemon balm may be present in a weight ratio from about 1:1 to about 25:1.

In some embodiments, the ginseng and lemon balm may be present in a weight ratio of from about 1:10 to about 10:1, such as from about 1:5 to about 10:1. In preferred embodiments, the ginseng and lemon balm may be present in a weight ratio of from about 1:1 to about 10:1.

In some embodiments, the vitamin C and lemon balm may be present in a weight ratio of from about 1:10 to about 10:1, such as from about 1:5 to about 10:1. In preferred embodiments, the vitamin C and lemon balm may be present in a weight ratio of from about 1:1 to about 10:1.

In some embodiments, the Echinacea and lemon balm may be present in a weight ratio of from about 1:5 to about 50:1, such as from about 1:5 to about 40:1. In preferred embodiments, the Echinacea and lemon balm may be present in a weight ratio of from about 1:2 to about 30:1.

In some embodiments the L-theanine and chamomile may be present in a weight ratio of from about 1:1 to about 100:1, such as from about 2:1 to about 80:1. In preferred embodiments, the L-theanine and chamomile may be present in a weight ratio of from about 5:1 to about 75:1.

In some embodiments, the ginseng and chamomile may be present in a weight ratio of from about 1:1 to about 25:1, such as from about 1:1 to about 20:1. In preferred embodiments, the ginseng and chamomile may be present in a weight ratio of from about 2:1 to about 20:1.

In some embodiments, the vitamin C and chamomile may be present in a weight ratio of from about 1:1 to about 50:1, such as about 2:1 to about 40:1. In preferred embodiments, the vitamin C and chamomile may be present in a weight ratio of from about 5:1 to about 25:1.

In some embodiments, the Echinacea and chamomile may be present in a weight ratio of about 1:1 to about 150:1, such as about 1:1 to about 120:1. In preferred embodiments, the Echinacea and chamomile may be present in a weight ratio of about 2:1 to about 100:1.

The skilled person will understand that any of the above weight ratio ranges may be combined.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:10 to about 35:1; wherein the weight ratio of vitamin C to L-theanine is from about 1:10 to about 10:1; and wherein the weight ratio of L-theanine to Echinacea is from about 1:10 to about 25:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:5 to about 30:1; wherein the weight ratio of vitamin C to L-theanine is from about 1:10 to about 10:1; and wherein the weight ratio of L-theanine to Echinacea is from about 1:10 to about 25:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:1 to about 25:1; wherein the weight ratio of vitamin C to L-theanine is from about 1:10 to about 10:1; and wherein the weight ratio of L-theanine to Echinacea is from about 1:10 to about 25:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:10 to about 35:1; wherein the weight ratio of vitamin C to L-theanine is from about 1:10 to about 8:1; and wherein the weight ratio of L-theanine to Echinacea is from about 1:10 to about 25:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:10 to about 35:1; wherein the weight ratio of vitamin C to L-theanine is from about 1:10 to about 5:1; and wherein the weight ratio of L-theanine to Echinacea is from about 1:10 to about 25:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:5 to about 30:1; wherein the weight ratio of vitamin C to L-theanine is from about 1:10 to about 8:1; and wherein the weight ratio of L-theanine to Echinacea is from about 1:10 to about 25:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:5 to about 30:1; wherein the weight ratio of vitamin C to L-theanine is from about 1:10 to about 8:1; and wherein the weight ratio of L-theanine to Echinacea is from about 1:10 to about 20:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:5 to about 30:1; wherein the weight ratio of vitamin C to L-theanine is from about 1:10 to about 8:1; and wherein the weight ratio of L-theanine to Echinacea is from about 1:5 to about 15:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:1 to about 30:1; wherein the weight ratio of vitamin C to L-theanine is from about 1:10 to about 8:1; and wherein the weight ratio of L-theanine to Echinacea is from about 1:10 to about 25:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:1 to about 30:1; wherein the weight ratio of vitamin C to L-theanine is from about 1:10 to about 5:1; and wherein the weight ratio of L-theanine to Echinacea is from about 1:10 to about 25:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:1 to about 30:1; wherein the weight ratio of vitamin C to L-theanine is from about 1:10 to about 5:1; and wherein the weight ratio of L-theanine to Echinacea is from about 1:5 to about 15:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:1 to about 25:1; wherein the weight ratio of vitamin C to L-theanine is from about 1:10 to about 5:1; and wherein the weight ratio of L-theanine to Echinacea is from about 1:5 to about 15:1.

In any of the above embodiments, the combination of active ingredients may be defined by one or more further weight ratios as defined above.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:10 to about 35:1; wherein the weight ratio of vitamin C to ginseng is from about 1:10 to about 10:1; and wherein the weight ratio of ginseng to Echinacea is from about 1:25 to about 5:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:5 to about 30:1; wherein the weight ratio of vitamin C to ginseng is from about 1:10 to about 10:1; and wherein the weight ratio of ginseng to Echinacea is from about 1:25 to about 5:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:1 to about 25:1; wherein the weight ratio of vitamin C to ginseng is from about 1:10 to about 10:1; and wherein the weight ratio of ginseng to Echinacea is from about 1:25 to about 5:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:10 to about 35:1; wherein the weight ratio of vitamin C to ginseng is from about 1:5 to about 10:1; and wherein the weight ratio of ginseng to Echinacea is from about 1:25 to about 5:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:10 to about 35:1; wherein the weight ratio of vitamin C to ginseng is from about 1:5 to about 5:1; and wherein the weight ratio of ginseng to Echinacea is from about 1:25 to about 5:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:10 to about 35:1; wherein the weight ratio of vitamin C to ginseng is from about 1:10 to about 10:1; and wherein the weight ratio of ginseng to Echinacea is from about 1:25 to about 4:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:10 to about 35:1; wherein the weight ratio of vitamin C to ginseng is from about 1:10 to about 10:1; and wherein the weight ratio of ginseng to Echinacea is from about 1:20 to about 4:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:5 to about 30:1; wherein the weight ratio of vitamin C to ginseng is from about 1:10 to about 10:1; and wherein the weight ratio of ginseng to Echinacea is from about 1:25 to about 4:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:5 to about 30:1; wherein the weight ratio of vitamin C to ginseng is from about 1:5 to about 10:1; and wherein the weight ratio of ginseng to Echinacea is from about 1:25 to about 4:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:1 to about 25:1; wherein the weight ratio of vitamin C to ginseng is from about 1:5 to about 10:1; and wherein the weight ratio of ginseng to Echinacea is from about 1:25 to about 4:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:5 to about 30:1; wherein the weight ratio of vitamin C to ginseng is from about 1:5 to about 5:1; and wherein the weight ratio of ginseng to Echinacea is from about 1:25 to about 4:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:1 to about 25:1; wherein the weight ratio of vitamin C to ginseng is from about 1:5 to about 5:1; and wherein the weight ratio of ginseng to Echinacea is from about 1:25 to about 4:1.

In some embodiments, the oral product comprises a combination of active ingredients comprising: (i) L-theanine, (ii) ginseng, (iii) vitamin C, and (iv) Echinacea, wherein the weight ratio of L-theanine to ginseng is from about 1:1 to about 25:1; wherein the weight ratio of vitamin C to ginseng is from about 1:5 to about 5:1; and wherein the weight ratio of ginseng to Echinacea is from about 1:20 to about 4:1.

In any of the above embodiments, vitamin D may be present in the combination of active ingredients and the weight ratio of vitamin C to vitamin D may be about 150000:1 to about 1000:1, such as from about 120000:1 to about 2000:1. In preferred embodiments, the weight ratio of vitamin C to vitamin D is from about 7000:1 to about 2500:1.

In any of the above embodiments, the combination of active ingredients may further comprise selenium. When selenium is present, the vitamin C and selenium may be present in a weight ratio of about 10000000:1 to about 10:1. In preferred embodiments, the vitamin C and selenium may be present in a weight ratio of from about 100000:1 to about 100:1.

In any of the above embodiments, chamomile may be present in the combination of active ingredients and the weight ratio of L-theanine to chamomile may be about 1:1 to 100:1, such as about 2:1 to about 80:1, preferably about 5:1 to about 75:1. Lemon balm may also or alternatively be present in the combination of active ingredients and the weight ratio of L-theanine to lemon balm may be from about 1:10 to about 35:1, such as from about 1:5 to about 30:1, preferably from about 1:1 to about 25:1.

### Additives

The liquid oral product may include one or more additional components in addition to the combination of active ingredients. For example, the oral product may further comprise an additive selected from the group consisting of a flavouring agent, sweetener, buffering agent, acidifying agent, humectant, preservative, salt, colouring agent, oral care additive, antioxidant, water or mixtures thereof. In some embodiments, the oral product may further comprise water and one or more additives selected from the group consisting of a flavouring agent, sweetener, acidifying agent, preservative, and mixtures thereof.

### Water

The moisture content (e.g., water content) of the oral product, prior to use by a consumer of the product, may vary according to the desired properties.

For the liquid oral product, the water content may be at least about 50%, such as at least about 60%, such as at least about 75% by weight of the oral product. Preferably, the water content of the liquid oral product may be at least about 90% by weight of the oral product. Preferably, the water content of a liquid oral product may be from about 60% to about 99.5% by weight, such as from about 75% to about 99% by weight, such as from about 80% to about 98% by weight of the oral product.

### Flavouring Agent

As used herein, the term “flavouring agent” (or “flavour” or “flavourant”) refers to materials which, where local regulations permit, may be used to create a desired taste, aroma or other somatosensorial sensation in a product for adult consumers. Examples of sensory characteristics that can be modified by the flavoring agent include taste, mouthfeel, moistness, coolness/heat, and/or fragrance/aroma. Flavouring agents may be natural or synthetic, and the character of the flavours imparted thereby may be described, without limitation, as fresh, sweet, herbal, confectionary, floral, fruity, or spicy.

The flavouring agent may be selected from the group consisting of naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, raspberry, strawberry, peach, apple, orange, mango, pineapple, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, coconut, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon basil, chive, carvi, verbena, tarragon, limonene, thymol, camphene), flavour enhancers, bitterness receptor site blockers, sensorial receptor site activators or stimulators, sugars and/or sugar substitutes (e.g., honey, sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavouring agent comprises a natural flavoring, such as berry (e.g. raspberry, blueberry or strawberry), honey, citrus (such as lemon, bergamot, orange or lime), or other botanical material.

In some embodiments, the flavouring agent comprises menthol, spearmint and/or peppermint. In some embodiments, the flavouring agent comprises flavour components of cucumber, blueberry, citrus fruits and/or redberry. In some embodiments, the flavouring agent comprises eugenol. In some embodiments, the flavouring agent comprises flavour components extracted from tobacco. In some embodiments, the flavouring agent comprises flavour components extracted from Cannabis.

In some embodiments, the flavouring agent may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-3.

In some embodiments, the flavouring agent may comprise a terpene. In some embodiments, the flavouring agent may comprise a monoterpene and/or a diterpene and/or a sesquiterpene. In some embodiments, the flavouring agent may comprise a monoterpene. In some embodiments, the terpene is selected from pinene (alpha and beta), geraniol, linalool, limonene, carvone, eucalyptol, menthone, iso-menthone, piperitone, myrcene, beta-bourbonene, germacrene, thymol, citral, eugenol, and mixtures thereof. In some embodiments, the flavouring agent is selected from the group consisting of geraniol, citronellol, nerol, maltol, ethylmaltol, fenchol, homofuraneol, furaneol, norfuraneol, 1-octen-3-ol, borneol, linalool, farnesol, hydroxycitronellol, 3,7-dimethyloctanol, myrcenol, lavandulol, nerolidol, terpineol, alpha-terpineol, menthol, thymol, carvacrol, myrtenol, carveol, santalol, piperitol, perillyl alcohol, patchouli alcohol, hexanol, 1-hexanol, 3-cis-hexanol, cis-3-hexen-1-ol, phenylethanol, eugenol, sesamol, sotolone, maple furanone, methyl anthranilate, guaiacol, raspberry ketone, 2-methoxy-4-vinylphenol, 4-ethylguaiacol, benzylalcohol, homofuraneol, vanillin, ethylvanillin, and combinations thereof. In some embodiments, the flavouring agent is selected from the group consisting of geraniol, citronellol, nerol, maltol, ethylmaltol, fenchol, homofuraneol, f furaneol, 1-octen-3-ol, borneol, norfuraneol, linalool, farnesol, hydroxycitronellol, 3,7-dimethyloctanol, myrcenol, lavandulol, nerolidol, terpineol, alpha-terpineol, menthol, thymol, carvacrol, myrtenol, carveol, santalol, piperitol, perillyl alcohol, patchouli alcohol, hexanol, 1-hexanol, 3-cis-hexanol, cis-3-hexen-1-ol, phenylethanol, eugenol, sesamol, sotolone, maple furanone, methyl anthranilate, guaiacol, raspberry ketone, 2-methoxy-4-vinylphenol, 4-ethylguaiacol, benzylalcohol, homofuraneol, and combinations thereof.

Where present, a flavouring agent may be included in the oral product in an amount up to about 10% by weight, such as up to about 5% by weight, such as up to about 1% by weight of the oral product. In some embodiments, a flavouring agent is present in an amount of from about 0.01% to about 5% by weight, such as in an amount of from about 0.1% to about 2.5% by weight of the oral product, preferably in an amount of from about 0.25% to about 1% by weight of the oral product.

### Humectants

In some embodiments, the oral product comprises at least one humectant. Examples of suitable humectants that may be included in the product include, but are not limited to, glycerin, 1,2-propanediol (propylene glycol), 1,3-propanediol, dipropylene glycol, sorbitol, xylitol, mannitol, and the like. In some embodiments, the humectant is or comprises glycerin. In some embodiments, the oral product comprises glycerin. In some embodiments, the humectant is or comprises propylene glycol. In some embodiments, the oral product comprises propylene glycol. The amount of humectant utilised in the oral product can vary, but may be up to about 5% by weight, and certain embodiments can be characterised by a humectant content of at least about 1% by weight, such as about 2 to about 5% by weight of the oral product. In some embodiments, the humectant (such as glycerin and/or propylene glycol) may be present in an amount of from about 0.01% to about 25% by weight of the oral product, such as from about 0.1% to about 20% by weight of the oral product, such as from about 0.5% to about 15% by weight of the oral product, such as from about 1% to about 10% by weight of the oral product, such as from about 5% to about 10% by weight of the oral product.

### Sweeteners

In order to improve the sensory properties of the oral product, one or more sweeteners may be added. The sweeteners can be any sweetener or combination of sweeteners, in natural or artificial form, or as a combination of natural and artificial sweeteners. Examples of natural sweeteners include fructose, sucrose, glucose, maltose, mannose, galactose, lactose, stevia, honey, and the like. Examples of artificial sweeteners include sucralose, isomaltulose, maltodextrin, saccharin, aspartame, acesulfame K, neotame and the like.

In some embodiments, the sweetener comprises one or more sugar alcohols. The sugar alcohol may include erythritol, arabitol, ribitol, isomalt, maltitol, dulcitol, iditol, mannitol, xylitol, lactitol, sorbitol, or combinations thereof. In some embodiments, the sweetener is selected from the group consisting of fructose, sucrose, glucose, maltose, mannose, galactose, lactose, stevia, honey, sucralose, isomaltulose, maltodextrin, saccharin, aspartame, acesulfame K, neotame, erythritol, arabitol, ribitol, isomalt, maltitol, dulcitol, iditol, mannitol, xylitol, lactitol, sorbitol, and mixtures thereof.

In some embodiments, the sweetener is selected from the group consisting of sucralose, acesulfame K, aspartame, maltodextrin, mannitol, sucrose, and mixtures thereof. Preferably, the sweetener may be sucralose and/or acesulfame K. When present in the oral product, the sweetener (such as sucralose and/or acesulfame K) may be present in an amount of from about 0.001% to about 5% by weight, such as from about 0.01% to about 3% by weight, preferably from about 0.01% to about 1% by weight of the oral product.

### Buffering Agents

Non-limiting examples of suitable buffering agents that may be included in the oral product include alkali metals acetates, glycinates, phosphates, glycerophosphates, citrates, carbonates, hydrogen carbonates, borates, or mixtures thereof. In some embodiments, in which a buffering agent is present, the buffering agent is selected from the group consisting of sodium carbonate, sodium bicarbonate, sodium phosphate, ammonium phosphate, dicalcium phosphate, tricalcium phosphate, and mixtures thereof. In some embodiments, the buffering agent is sodium bicarbonate and/or sodium carbonate. Where present, the buffering agent (e.g. sodium bicarbonate and/or sodium carbonate) may be included in an amount less than about 5% based on the weight of the oral product; for example, from about 0.5% to about 5%, such as, e.g., from about 0.75% to about 4%, from about 0.75% to about 3%, or from about 1% to about 2% by weight, based on the total weight of the oral product.

### Organic Acid

In some embodiments, the product comprises an organic acid. As used herein, the term “organic acid” refers to an organic (i.e., carbon-based) compound that is characterised by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific mixture ingredient as opposed to merely being inherently present as a component of another mixture ingredient (e.g., the small amount of organic acid which may inherently be present in a mixture ingredient such as a tobacco material). In some embodiments, the one or more organic acids are added neat (i.e., in their free acid, native solid or liquid form) or as a solution in, e.g. water. In some embodiments, the one or more organic acids are added in the form of a salt.

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

In some embodiments, the organic acid is an alkyl carboxylic acid. Non-limiting examples of alkyl carboxylic acids include formic acid, acetic acid, propionic acid, octanoic acid, nonanoic acid, decanoic acid, undecanoic acid, dodecanoic acid, stearic acid, oleic acid, linoleic acid, linolenic acid, and the like. In some embodiments, the organic acid is an alkyl sulfonic acid. Non-limiting examples of alkyl sulfonic acids include propanesulfonic acid and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like. In some preferred embodiments, the organic acid is citric acid, sodium citrate, calcium citrate, or a combination thereof.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Additional non-limiting examples of suitable organic acids include 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), camphoric acid (+), camphor-10-sulfonic acid (+), capric acid, caproic acid, caprylic acid, cinnamic acid, cyclamic acid, decanoic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glycerophosphoric acid, glycolic acid, hippuric acid, isobutyric acid, lactobionic acid, lauric acid, malonic acid, mandelic acid, methanesulfonic acid, naphthalene-1,5-disulfonic acid, naphthalene-2-sulfonic acid, oleic acid, palmitic acid, pamoic acid, pyroglutamic acid, sebacic acid, stearic acid, and undecylenic acid.

Preferably, the organic acid is selected from the group consisting of citric acid, malic acid, lactic acid, benzoic acid, tartaric acid, and mixtures thereof. In some preferred embodiments, the organic acid is or comprises citric acid or a salt thereof.

In some embodiments, the product comprises an alkali metal salt of an organic acid. For example, at least a portion of the organic acid may be present in the product in the form of an alkali metal salt. Suitable alkali metal salts include lithium, sodium, and potassium. In some embodiments, the alkali metal is sodium or potassium. In some embodiments, the alkali metal is sodium. In some embodiments, the product comprises an organic acid and a sodium salt of the organic acid. In some embodiments, the organic acid is or comprises sodium citrate, such as trisodium citrate.

The amount of organic acid present in the product may vary. The oral product may comprise from about 0.01% to about 10% by weight of organic acid, present as one or more organic acids, based on the total weight of the oral product. In some embodiments, the oral product comprises at least about 0.01%, at least about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or at least about 10% organic acid by weight, based on the total weight of the oral product. In some preferred embodiments, the oral product comprises from about 0.01% to about 5% by weight of organic acid based on the weight of the oral product. For example, the oral product comprises an organic acid in an amount of from about 0.1% to about 2.5% by weight of the oral product. In the case where a salt of an organic acid is added (e.g., citric acid anhydrate), the percent by weight is calculated based on the weight of the free acid, not including any counter-ion which may be present.

In certain embodiments the organic acid inclusion is sufficient to provide a product pH of from about 4.0 to about 9.0, such as from about 4.5 to about 7.0, or from about 5.5 to about 7.0, from about 4.0 to about 5.5, or from about 7.0 to about 9.0. In some embodiments, the organic acid inclusion is sufficient to provide a product pH of from about 4.5 to about 6.5, for example, from about 4.5, about 5.0, or about 5.5, to about 6.0, or about 6.5. In some embodiments, the organic acid is provided in a quantity sufficient to provide a pH of the product of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In other embodiments, a mineral acid (e.g., hydrochloric acid, sulfuric acid, phosphoric acid, or the like) is added to adjust the pH of the product to the desired value.

Organic acids (e.g., citric acid) may be added neat (i.e., as a solid) or in solution, for example, in water. In some embodiments, the organic acid is added as a 50% aqueous solution.

### Salts

The oral product may further comprise a salt. This may be included in an amount sufficient to provide desired sensory attributes to the product. Non-limiting examples of suitable salts include sodium chloride, potassium chloride, ammonium chloride, flour salt, sodium acetate, sodium citrate, and the like. The salt may be included in any suitable amount, such as at least about 0.5% by weight, such as at least about 1% by weight, such as at least about 1.5% by weight of the oral product. In some embodiments, the oral product may comprise salt in an amount of from about 0.5% to about 10% by weight, such as from about 1% to about 7.5% by weight, such as from about 1.5% to about 5% by weight, based on the total weight of the oral product.

### Colouring Agents

A colouring agent may be employed in amounts sufficient to provide the desired physical attributes to the product. Examples of colouring agents include various dyes and pigments, such as caramel colouring and titanium dioxide. Natural colouring agents such as curcumin, beet juice extract, spirulina; also a variety of synthetic pigments may also be used. The amount of colorant utilised in the oral product can vary, but when present is typically up to about 3% by weight, such as from about 0.1%, about 0.5%, or about 1%, to about 3% by weight, based on the total weight of the oral product.

### Other Additives

Other ingredients such as preservatives (e.g., potassium sorbate), and/or antioxidants can also be used. Typically, such ingredients, where used, are used in amounts of up to about 10% by weight, for example at least about 0.1% by weight, such as about 0.5 to about 10% by weight of the oral product.

Examples of further types of additives include zinc or magnesium salts selected to be relatively water soluble for compositions with greater water solubility (e.g., magnesium or zinc gluconate) or selected to be relatively water insoluble for compositions with reduced water solubility (e.g., magnesium or zinc oxide), or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No 2007/0062549 to Holton, Jr. et al., each of which is incorporated herein by reference. Typical inclusion ranges for such additional additives can vary depending on the nature and function of the additive and the intended effect on the final product, with an example range of up to about 10% by weight, based on total weight of the oral product (e.g., about 0.1 to about 5% by weight).

In some embodiments, the oral product comprises a magnesium salt. A non-limiting example of a suitable magnesium salt is magnesium gluconate. In some embodiments, the oral product comprises magnesium in an amount by weight from about 0.1% to about 2%, or from about 0.2 to about 1%, based on elemental magnesium.

The aforementioned additives can be employed together (e.g., as additive formulations) or separately (e.g., individual additive components can be added at different stages involved in the preparation of the final product).

### Oral Product

The products or compositions as described herein are configured for oral use. The term “configured for oral use” as used herein means that the product is provided in a form such that during use, saliva in the mouth of the user causes one or more of the components of the product (e.g., active ingredients) to pass into the mouth of the user. In certain embodiments, the product is adapted to deliver active ingredients and optionally flavouring agent to a user through mucous membranes in the user's mouth, the user's digestive system, or both.

The combination of active ingredients may be present in an amount of from about 0.01% to about 20% by weight. For example, the combination of active ingredients may be present in an amount of from about 0.05% to about 15% by weight, such as from about 0.1% to about 10% by weight. Preferably, the combination of active ingredients is present in an amount of from about 0.1% to about 15% by weight of the oral product, more preferably from about 1% to about 10% by weight of the oral product.

The liquid oral product may be in the form of a shot; i.e. a drink that may be consumed quickly, e.g. in a single gulp or a couple of gulps. Formulating the oral product in the form of a shot may be advantageous as it provides a convenient and easy-to-use mode of administration that does not take up much room during storage (e.g. in a fridge or cupboard) and is easily portable by the user so can be taken on the go. Shots may also provide an efficient delivery vehicle for actives, whereby a level of active sufficient to provide good efficacy can be delivered to the user without the need to consume large volumes of liquid. Additionally, the shots may be formulated to have a pleasant taste. Drinking a shot may be considered to be more palatable and easier to a user than swallowing a tablet, for example.

The liquid oral product may also be in the form of a larger beverage that is consumed more slowly over several gulps. The liquid oral product may have a volume of from about 1 mL to about 250 mL, such as from about 1 mL to about 200 mL. The liquid oral product may have a volume of from about 100 mL to about 250 mL and be provided in a package such as a carton, cup, can or bottle of liquid.

The liquid oral product may have a volume of from about 10 mL to about 100 mL, such as from about 25 mL to about 75 mL. In such embodiments, the liquid oral product may be considered to be a shot. For example, the volume of liquid may be about 60 mL or about 30 mL.

As noted above, the oral product may further comprise water in an amount of from about 50% to about 99.9% by weight of the oral product. In some embodiments, the oral product comprises water in an amount of from about 75% to about 99.5% by weight, such as from about 80% to about 99% by weight, such as from about 90% to about 97.5% by weight of the oral product. In some embodiments, the oral product comprises water in an amount of from about 90% to about 99.5% by weight of the oral product, and may be from about 95% to about 99% by weight of the oral product.

Where present, the water may include tap water, rain water, mineralised water, or distilled water.

The liquid oral product may comprise the combination of active ingredients in an amount of from about 0.1% to about 15% by weight of the oral product, and water in an amount of from about 85% to about 99.9% by weight of the oral product. The liquid oral product may comprise the combination of active ingredients in an amount of from about 0.5% to about 12% by weight of the oral product, and water in an amount of from about 88% to about 99.5% by weight of the oral product.

In any of the above embodiments, the liquid oral product may further comprise zinc or zinc gluconate, optionally in an amount of from about 0.001% to about 2% by weight of the oral product. In preferred embodiments, zinc or zinc gluconate may be present in an amount of about 0.005% to about 1% by weight of the oral product.

The liquid oral product (e.g. in the form of a shot having a volume of 25 mL to 75 mL) may comprise L-theanine in an amount of from about 25 mg to about 2000 mg, such as from about 50 mg to about 1800 mg, preferably from about 75 mg to about 1500 mg.

The liquid oral product (e.g. in the form of a shot having a volume of 25 mL to 750 mL) may comprise ginseng in an amount of from about 10 mg to about 500 mg, such as from about 25 mg to about 300 mg, preferably from about 50 mg to about 250 mg.

The liquid oral product (e.g. in the form of a shot having a volume of 25 mL to 75 mL) may include vitamin C in an amount of from about 10 mg to about 500 mg or from about 50 mg to about 500 mg, such as from about 100 mg to about 500 mg, preferably from about 200 mg to about 400 mg.

The liquid oral product (e.g. in the form of a shot having a volume of 25 mL to 75 mL) may include Echinacea in an amount of from about 10 mg to about 1500 mg, such as from about 25 mg to about 1500 mg, preferably from about 40 mg to about 1000 mg.

When present, the liquid oral product (e.g. in the form of a shot having a volume of 25 mL to 75 mL) may include vitamin D in an amount of from about 0.001 mg to about 50 mg or from about 0.001 mg to about 1 mg, such as from about 0.001 mg to about 0.75 mg, preferably from about 0.0025 mg to about 0.5 mg.

When present, the liquid oral product (e.g. in the form of a shot having a volume of 25 mL to 75 mL) may include chamomile (e.g. chamomile extract) in an amount of from about 1 mg to about 100 mg, such as from about 5 mg to about 75 mg, preferably from about 10 mg to about 50 mg.

When present, the liquid oral product (e.g. in the form of a shot having a volume of 25 mL to 75 mL) may include lemon balm (e.g. lemon balm extract) in an amount of from about 1 mg to about 200 mg, such as from about 5 mg to about 150 mg, preferably from about 10 mg to about 120 mg.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 10 mg to about 1500 mg of Echinacea; (v) from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 10 mg to about 2500 mg of Echinacea; (v) from about 0.001 mg to about 1 mg of vitamin D; and (vi) from about 1 mg to about 100 mg of chamomile extract and optionally (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 10 mg to about 1500 mg of Echinacea; (v) from about 0.001 mg to about 1 mg of vitamin D; and (vi) from about 1 mg to about 100 mg of chamomile extract and (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 10 mg to about 500 mg of ginseng; (iii) from about 50 mg to about 500 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 10 mg to about 500 mg of ginseng; (iii) from about 50 mg to about 500 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 50 mg to about 500 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 25 mg to about 300 mg of ginseng; (iii) from about 50 mg to about 500 mg of vitamin C; (iv) from about 10 mg to about 2500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 50 mg to about 250 mg of ginseng; (iii) from about 50 mg to about 500 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2500 mg of L-theanine; (ii) from about 10 mg to about 500 mg of ginseng; (iii) from about 100 mg to about 500 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2500 mg of L-theanine; (ii) from about 10 mg to about 500 mg of ginseng; (iii) from about 200 mg to about 400 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 10 mg to about 500 mg of ginseng; (iii) from about 200 mg to about 400 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 200 mg to about 400 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 100 mg to about 500 mg of vitamin C; (iv) from about 10 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 25 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 40 mg to about 1000 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 10 mg to about 500 mg of ginseng; (iii) from about 50 mg to about 500 mg of vitamin C; (iv) from about 25 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 50 mg to about 500 mg of vitamin C; (iv) from about 25 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 100 mg to about 500 mg of vitamin C; (iv) from about 25 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 25 mg to about 300 mg of ginseng; (iii) from about 100 mg to about 500 mg of vitamin C; (iv) from about 25 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 50 mg to about 200 mg of ginseng; (iii) from about 100 mg to about 500 mg of vitamin C; (iv) from about 25 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 50 mg to about 200 mg of ginseng; (iii) from about 200 mg to about 400 mg of vitamin C; (iv) from about 25 mg to about 1500 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 50 mg to about 200 mg of ginseng; (iii) from about 200 mg to about 400 mg of vitamin C; (iv) from about 40 mg to about 1000 mg of Echinacea; (v) optionally from about 0.001 mg to about 1 mg of vitamin D; and optionally (vi) from about 1 mg to about 100 mg of chamomile extract and/or (vii) from about 1 mg to about 200 mg of lemon balm extract.

In any of the above embodiments, vitamin D may be present in the recited amount of about 0.001 mg to about 1 mg. In other embodiments, vitamin D may be present in an amount of about 0.001 mg to about 50 mg. In such embodiments, vitamin D may further be present in an amount of about 0.001 mg to about 0.75 mg, preferably in an amount of about 0.0025 mg to about 0.5 mg.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 10 mg to about 1500 mg of Echinacea; (v) from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 10 mg to about 2500 mg of Echinacea; (v) from about 0.001 mg to about 0.2 mg of selenium; and (vi) from about 1 mg to about 100 mg of chamomile extract and optionally (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 10 mg to about 1500 mg of Echinacea; (v) from about 0.001 mg to about 0.2 mg of selenium; and (vi) from about 1 mg to about 100 mg of chamomile extract and (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 10 mg to about 500 mg of ginseng; (iii) from about 50 mg to about 500 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 10 mg to about 500 mg of ginseng; (iii) from about 50 mg to about 500 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 50 mg to about 500 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 25 mg to about 300 mg of ginseng; (iii) from about 50 mg to about 500 mg of vitamin C; (iv) from about 10 mg to about 2500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 50 mg to about 250 mg of ginseng; (iii) from about 50 mg to about 500 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2500 mg of L-theanine; (ii) from about 10 mg to about 500 mg of ginseng; (iii) from about 100 mg to about 500 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2500 mg of L-theanine; (ii) from about 10 mg to about 500 mg of ginseng; (iii) from about 200 mg to about 400 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 10 mg to about 500 mg of ginseng; (iii) from about 200 mg to about 400 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 200 mg to about 400 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 100 mg to about 500 mg of vitamin C; (iv) from about 10 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 25 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 25 mg to about 2000 mg of L-theanine; (ii) from about 10 mg to about 500 mg of ginseng; (iii) from about 50 mg to about 500 mg of vitamin C; (iv) from about 40 mg to about 1000 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 10 mg to about 500 mg of ginseng; (iii) from about 50 mg to about 500 mg of vitamin C; (iv) from about 25 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 50 mg to about 500 mg of vitamin C; (iv) from about 25 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 50 mg to about 1800 mg of L-theanine; (ii) from about 25 mg to about 300 mg of ginseng; (iii) from about 100 mg to about 500 mg of vitamin C; (iv) from about 25 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 25 mg to about 300 mg of ginseng; (iii) from about 100 mg to about 500 mg of vitamin C; (iv) from about 25 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 50 mg to about 200 mg of ginseng; (iii) from about 100 mg to about 500 mg of vitamin C; (iv) from about 25 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 50 mg to about 200 mg of ginseng; (iii) from about 200 mg to about 400 mg of vitamin C; (iv) from about 25 mg to about 1500 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In some embodiments, the liquid oral product (e.g. in the form of a shot having a volume of 25 mL to 75 mL) comprises: (i) from about 75 mg to about 1500 mg of L-theanine; (ii) from about 50 mg to about 200 mg of ginseng; (iii) from about 200 mg to about 400 mg of vitamin C; (iv) from about 40 mg to about 1000 mg of Echinacea; (v) optionally from about 0.001 mg to about 0.2 mg of selenium; and optionally (vi) from about 1 mg to about 100 mg of chamomile extract and/or (vii) from about 1 mg to about 200 mg of lemon balm extract.

In any of the above embodiments, selenium may be present in the recited amount of about 0.001 mg to about 0.2 mg. In such embodiments, selenium may further be present in an amount of about 0.005 mg to about 0.1 mg, preferably in an amount of about 0.01 mg to about 0.05 mg.

In any of the above embodiments, chamomile extract may be present in the recited amount of about 1 mg to about 100 mg. In such embodiments, chamomile extract may further be present in an amount of about 5 mg to about 75 mg, preferably in an amount of about 10 mg to about 50 mg.

In any of the above embodiments, lemon balm extract may be present in the recited amount of about 1 mg to about 200 mg. In such embodiments, lemon balm extract may further be present in an amount of about 5 mg to about 150 mg, preferably in an amount of about 10 mg to about 120 mg.

The amounts of the active ingredients as described above have been found by the inventors to provide a beneficial effect of boosting immune response, supporting immunity and/or resilience against infection, while providing a safe product with reduced side effects. The amounts may be tailored such that the product has high efficacy while also ensuring consumer safety and avoiding any overdose of actives.

In any of the above embodiments, the liquid oral product may further comprise zinc, such as zinc gluconate, optionally in an amount of from about 5 mg to about 150 mg. In preferred embodiments, the liquid oral product may further comprise zinc in an amount of from about 10 mg to about 50 mg.

The liquid oral product may include any further suitable additive. Suitable additives are described in greater detail above, and all of the additives described herein may be included in the liquid oral product. In some embodiments, the liquid oral product may further comprise an additive selected from the group consisting of a flavouring agent, sweetener, acidifying agent, humectant, preservative, and mixtures thereof. Examples of each of these forms of additives are described hereinabove.

In some embodiments, there is provided a package in the form of a bottle or can that contains the liquid oral product. The package may be a bottle containing the desired volume of liquid oral product.

### Process

The manner by which the various components of the composition (e.g., active ingredients and any additives) are combined may vary. The components noted above, which may be in liquid or dry solid form, can be admixed in a pre-treatment step prior to mixture with any remaining components of the product, or simply mixed together with all other liquid or dry ingredients.

The various components of the product may be contacted, combined, or mixed together using any mixing technique or equipment known in the art. Any mixing method that brings the product ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. Nos. 4,148,325 to Solomon et al.; U.S. Pat. No. 6,510,855 to Korte et al.; and U.S. Pat. No. 6,834,654 to Williams, each of which is incorporated herein by reference. Manners and methods for formulating products will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. No. 4,725,440 to Ridgway et al., and U.S. Pat. No. 6,077,524 to Bolder et al., each of which is incorporated herein by reference.

In accordance with some embodiments described herein, there is provided a process for preparing an oral product as described herein, the oral product preferably being in the form of a liquid, the process comprising the steps of:
(a) combining active ingredients,
(b) contacting the active ingredients with water, and
(c) mixing the active ingredients with water to prepare an oral product.

The combination of active ingredients may be as described hereinabove. The combination of actives may also be as described below in respect of “Further Broad Aspects”.

The active ingredients may be provided in the form of a liquid extract, a liquid oil or a powder. When in the form of a powder, step (c) may comprise mixing the active ingredients with water until said active ingredients have dissolved in the water.

Step (b) and/or step (c) may preferably be carried out at ambient or room temperature (20-25° C.). Alternatively, the temperature may be elevated in order to assist with dispersion or dissolution of the active ingredients in the water. For example, step (b) may comprise contacting the active ingredients with water at a temperature of from about 20-100° C., such as from about 30-90° C., or from about 40-80° C. Step (b) may comprise mixing the active ingredients with water at a temperature of from about 20-100° C., such as from about 30-90° C., or from about 40-80° C.

The process may comprise adding any additional additives at any stage. For example, any additives may be added to the active ingredients prior to combination with water and/or after the active ingredients have been combined with water. The additives may also be added to the water prior to contacting the water with the combination of active ingredients. Step (a) may comprise an optional step of combining any additives (e.g. acidifying agent) with the combination of active ingredients. Step (b) may comprise an optional step of contacting the active ingredients with water and an additive. Step (c) may comprise an optional step of adding additives to the mixture and mixing the additives with the combination of active ingredients and water.

In some embodiments, the oral product further comprises one or more additives. In such embodiments, the one or more additives may be combined with water prior to addition of the active ingredients. Optionally, the step of combining the one or more additives with water may comprise heating the combination, e.g. to a temperature of from about 60° C. to about 80° C., to achieve dissolution of the additive(s). Further additives, such as a sweetener, humectant, colouring agent or the like, may be added at this stage or may be added during and/or after the step of combining the active ingredients with the water.

The resulting liquid product may be in the form of a solution or dispersion of active ingredients in water. Preferably the liquid product is in the form of a solution of active ingredients in water.

### Use

According to some embodiments described herein, there is provided the use of a combination of active ingredients to boost immune response or support immunity of a human or animal, wherein the combination of active ingredients comprises (i) L-theanine; (ii) ginseng; (iii) vitamin C; (iv) Echinacea; and optionally vitamin D or selenium. In preferred embodiments, the combination of active ingredients is present in a liquid oral product according to the disclosure hereinabove.

In some embodiments, the combination of active ingredients further comprises any of the additional active ingredients as described hereinabove. For example, the combination of active ingredients may further comprise chamomile extract and/or lemon balm.

The combination of active ingredients may provide an improved effect in boosting immune response or supporting immunity as compared with previously known products or a placebo (i.e. an oral product which does not include the combination of active ingredients according to the present invention). Immune response and/or immunity may be measured by methods commonly known in the field, for example laboratory studies of patient samples such as blood/serum tests measuring e.g. immune cell populations and subpopulations, markers such as pro-inflammatory cytokines, and/or immunoglobulins.

According to some embodiments described herein, there is provided the use of a combination of active ingredients to support resilience of a human or animal against infection, wherein the combination of active ingredients comprises (i) L-theanine; (ii) ginseng; (iii) vitamin C; (iv) Echinacea; and optionally vitamin D or selenium. In preferred embodiments, the combination of active ingredients is present in a liquid oral product according to the disclosure hereinabove. Resilience to infection may be measured by methods commonly known in the field, for example human population studies/random controlled trials, and/or via laboratory studies of immune cells, markers, immunoglobulins etc. in patient samples.

In further embodiments described herein, there is provided the use of the combination of active ingredients according to the embodiments defined herein, to improve alertness and to improve feelings of being in control, of a human or animal. The improvement of alertness or feelings of being in control is compared to a placebo (i.e. an oral product which does not include the combination of active ingredients according to the present invention). The improvement of alertness may be assessed by a visual analogue mood scale (VAMS) as exemplified by Example 8 herein. The improvement of feelings of being in control may be assessed by a visual analogue scale (VAS) as exemplified by Example 8 herein. The human or animal may further be subject to a stressor (e.g. a Stroop test) during the assessments, as exemplified by Example 8 herein. Hence, the oral product of the present invention may be described as providing a mood state which is “more alert and in control”.

In further embodiments described herein, there is provided the use of the combination of active ingredients according to the embodiments defined herein, to reduce sleepiness of a human or animal. The reduction in sleepiness is compared to a placebo (i.e. an oral product which does not include the combination of active ingredients according to the present invention). The reduction in sleepiness may be assessed by a visual analogue scale (VAS) as exemplified by Example 8 herein.

In some embodiments, the combination of active ingredients further comprises any of the additional active ingredients as described hereinabove. For example, the combination of active ingredients may further comprise chamomile extract and/or lemon balm.

The combination of active ingredients may provide an improved effect in supporting resilience of a human or animal against infection as compared with previously known products or a placebo.

It will be understood that all disclosure herein concerning the liquid oral product applies to the disclosed process and use. The disclosure is not repeated for conciseness.

### Further Broad Aspects

In accordance with some embodiments described herein, there is provided a liquid oral product comprising a combination of active ingredients, wherein the combination of active ingredients comprises ginseng; vitamin C; and lemon balm, wherein the ginseng and lemon balm are present in a weight ratio of from about 1:10 to about 35:1. In some embodiments the lemon balm is present in an amount of from about 1 mg to about 200 mg.

Such oral product may further comprise other active ingredients. The active ingredients and/or additives described hereinabove are equally applicable to such embodiments and are only not repeated here for conciseness. All amounts and combinations described hereinabove equally apply to this embodiment.

In some embodiments, the amount of ginseng is from about 10 mg to about 500 mg.

Also described herein is a liquid oral product comprising a combination of active ingredients, wherein the combination of active ingredients comprises vitamin C, Echinacea and chamomile extract, wherein the weight ratio of Echinacea to chamomile is from about 1:1 to about 100:1, such as from about 1:1 to about 50:1, preferably from about 1:1 to about 20:1.

In any of the above embodiments, the oral product may further comprise an additional active ingredient as described hereinabove with respect to the first aspect. The ranges and combinations described hereinabove of these additional active ingredients equally apply to these embodiments.

The processes and uses described hereinabove may also equally apply to the above further broad aspects.

### EXAMPLES

Aspects of the present invention are more fully illustrated by the following examples, which are set forth to illustrate certain aspects of the present invention and are not to be construed as limiting thereof.

### Example 1—Oral Product—Liquid Dosage Form

An oral product in the form of a liquid is prepared containing the following ingredients per serving:

| Ingredients | Percentage amount (wt %) |
|---|---|
| Water | 85-99 |
| L-theanine | 0.01-15 |
| Vitamin C | 0.01-10 |
| Echinacea | 0.001-20 |
| Vitamin D | 0-0.01 |
| Ginseng extract | 0.01-5 |

The oral product has a volume of 30 or 60 mL per serving.

All active ingredients are added to the water, and the resulting mixture stirred until a homogeneous dispersion or clear solution is obtained. The product is then packaged in bottles, with each serving containing 30 or 60 mL of liquid.

An oral product in the form of a liquid is also prepared containing the following ingredients per serving:

| Ingredients | Percentage amount (wt %) |
|---|---|
| Water | 85-99 |
| L-theanine | 0.01-15 |
| Vitamin C | 0.01-10 |
| Echinacea | 0.001-20 |
| Vitamin D | 0-0.1 |
| Ginseng extract | 0.01-5 |

The oral product has a volume of 30 or 60 mL per serving.

All active ingredients are added to the water, and the resulting mixture stirred until a homogeneous dispersion or clear solution is obtained. The product is then packaged in bottles, with each serving containing 30 or 60 mL of liquid.

### Example 2—Liquid Dosage Form Containing 15:1 Ratio of L-Theanine:Ginseng

An oral product in the form of a liquid is prepared containing the following ingredients:
Water
L-theanine
Vitamin C
Ginseng extract
Echinacea
Vitamin D
Lemon balm

The amount of L-theanine is about 600 mg per serving and the amount of ginseng is about 40 mg per serving. The amount of vitamin C is about 200 mg per serving and the amount of vitamin D about 0.01 mg per serving. The amount of Echinacea is about 200 mg per serving and the amount of lemon balm is about 40 mg per serving. The oral product has a volume of 60 mL per serving.

The oral product is prepared as set out in Example 1.

### Example 3—Liquid Dosage Form Containing 2:1 Ratio of Vitamin C:Ginseng

An oral product in the form of a liquid is prepared containing the following ingredients:
Water
L-theanine
Vitamin C
Ginseng extract
Echinacea
Vitamin D
Chamomile extract

The amount of L-theanine is about 300 mg per serving. The amount of vitamin C is about 400 mg per serving and the amount of ginseng is about 200 mg per serving. The amount of Echinacea is about 100 mg per serving and the amount of chamomile extract is about 30 mg per serving. The amount of vitamin D is about 0.001 mg per serving. The oral product has a volume of 60 mL per serving.

The oral product is prepared as set out in Example 1.

### Example 4—Oral Product—Liquid Dosage Form Containing Selenium

An oral product in the form of a liquid is prepared containing the following ingredients per serving:

| Ingredients | Percentage amount (wt %) |
|---|---|
| Water | 85-99 |
| L-theanine | 0.01-15 |
| Vitamin C | 0.001-10 |
| Echinacea | 0.001-20 |
| Selenium | 0-0.001 |
| Ginseng extract | 0.01-5 |

The oral product has a volume of 60 mL per serving.

All active ingredients are added to the water, and the resulting mixture stirred until a homogeneous dispersion or clear solution is obtained. The product is then packaged in bottles, with each serving containing 60 mL of liquid.

### Example 5—Liquid Dosage Form Containing 15:1 Ratio of L-Theanine:Ginseng

An oral product in the form of a liquid is prepared containing the following ingredients:
Water
L-theanine
Vitamin C
Ginseng extract
Echinacea
Selenium
Lemon balm extract

The amount of L-theanine is about 600 mg per serving and the amount of ginseng is about 40 mg per serving. The amount of vitamin C is about 200 mg per serving and the amount of selenium about 0.02 mg per serving. The amount of Echinacea is about 200 mg per serving and the amount of lemon balm is about 40 mg per serving. The oral product has a volume of 60 mL per serving.

The oral product is prepared as set out in Example 4.

### Example 6—Liquid Dosage Form Containing 3:1 Ratio of Vitamin C:Ginseng

An oral product in the form of a liquid is prepared containing the following ingredients:
Water
L-theanine
Vitamin C
Ginseng extract
Echinacea
Selenium
Chamomile extract
Lemon balm extract

The amount of L-theanine is about 200 mg per serving. The amount of vitamin C is about 300 mg per serving and the amount of ginseng is about 100 mg per serving. The amount of Echinacea is about 100 mg per serving and the amount of chamomile extract about 30 mg per serving. The amount of selenium is about 0.02 mg per serving and the amount of lemon balm extract about 60 mg per serving.

The oral product has a volume of 60 mL per serving.

The oral product is prepared as set out in Example 4.

### Example 7—Liquid Dosage Form Containing 3:1 Ratio of Vitamin C:Ginseng

An oral product in the form of a liquid is prepared containing the following ingredients:
Water
L-theanine
Vitamin C
Ginseng extract
Echinacea
Vitamin D
Chamomile extract
Lemon balm extract

The amount of L-theanine is about 200 mg per serving. The amount of vitamin C is about 300 mg per serving and the amount of ginseng is about 100 mg per serving. The amount of Echinacea is about 40 mg per serving and the amount of chamomile extract about 20 mg per serving. The amount of vitamin D is about 0.001 mg per serving and the amount of lemon balm extract about 60 mg per serving.

The oral product has a volume of 60 mL per serving.

The oral product is prepared as set out in Example 4.

### Example 8—Results of Consumer Testing

A study was carried out to compare an oral product as prepared according to Example 7 with a placebo (shot without actives). Blind testing was carried out where one group of participants consumed the shot of Example 7, while a second group of participants were given the placebo product. The study investigated the acute effects approximately 2 hours post-consumption, of the liquid dosage form on measurements of alertness, feelings of control, and sleepiness. The measurements of alertness and feelings of control were made after completion of a controlled emotionally activating (mildly stressful/frustrating/challenging) computer game.

The following factors were assessed:

| Endpoint | Method |
|---|---|
| Alertness | Visual Analogue |
| | Mood Scale (VAMS) |
| Calm/sleepiness/nervousness/‘in control’ | Visual Analogue |
| | Scale (VAS) |

The participants in the study (n=48, across Example 7 and placebo groups) were aged between 29-45 years old and were considered to be healthy adults. The inclusion criteria included willingness to participate and self-reported good health (using the Zung scale). Main exclusion criteria included: a Zung scale value of >59, nicotine and CBD use, allergies listed within the formulation, heavy caffeine consumers (>400 mg/day), heavy alcohol consumers (>14 units/week), recreational and prescription drug users, pregnancy or breastfeeding/expressing, self-reported moderate-severe anxiety/depression, self-reported sleep disturbances/working night shifts, and presence of life events likely to induce unstable/fluctuating emotional states (e.g. change of profession, moving house, foreign holiday, divorce, surgery). The participants refrained from any food and drink within 1 hour before testing. Baseline measurements were carried out before administration of any product. These baseline measurements were made 5 minutes after the participant arrived at the study (i.e. with a 5 minute waiting period). The baseline measurements were of the end points above. Each participant then consumed one of the oral products; the products were tested in a double-blind approach.

After consumption, the participants were shown a documentary for 45 minutes before being asked to complete the same test methods as at baseline. After the mid-point measurements, the participants were shown a second documentary for 40 minutes and then asked to again complete the test methods before being subjected to a stressor (computer game to induce controlled emotional activation (Stroop Test)). Immediately after the stressor, measurements were taken of the end points and again in two rounds around 15 minutes and 30 minutes after the stressor. The participants were shown the documentaries between each measurement point.

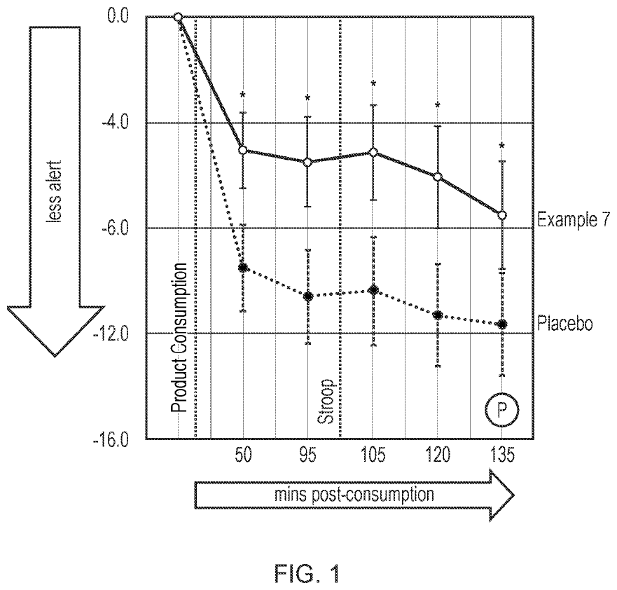

The results of the study are shown in FIGS. 1 to 5.

As can be seen from said results, participants given the liquid dosage form of Example 7 reported greater alertness, feeling in control and reduced sleepiness versus the placebo group. The placebo results are marked with “P” in the Figures. Without wishing to be bound by theory, it is believed that the combination of active ingredients as defined herein allows for these effects to be achieved.

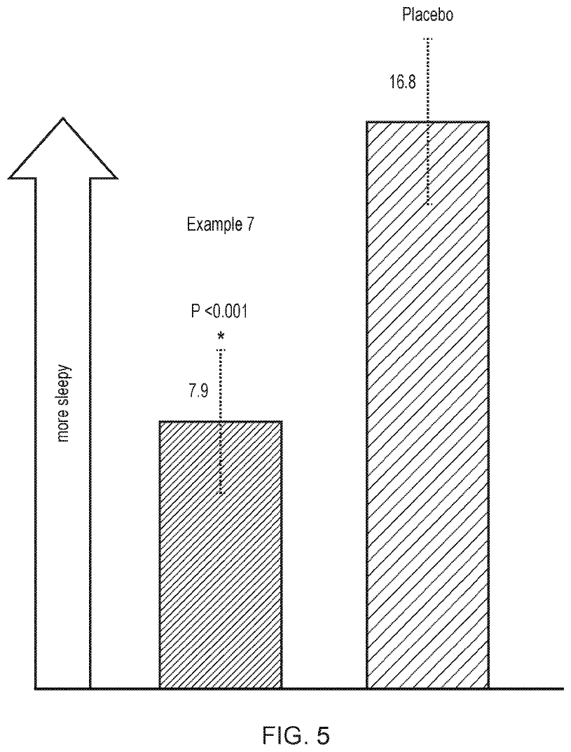

Perceived sleepiness was measured by via a visual analogue scale (VAS). The oral product of Example 7 showed significantly less tiredness/sleepiness compared with the placebo. This reduced tiredness was statistically different to the placebo over the time course of the study. The results are shown in FIG. 5 which shows the overall result for all post-consumption time-points combined.

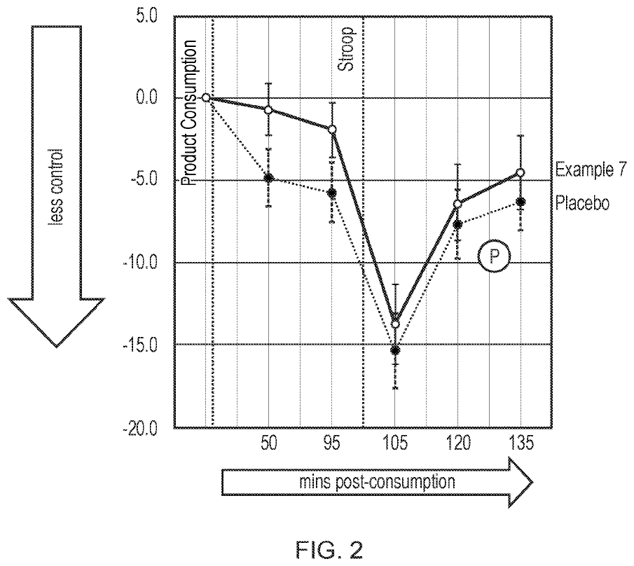

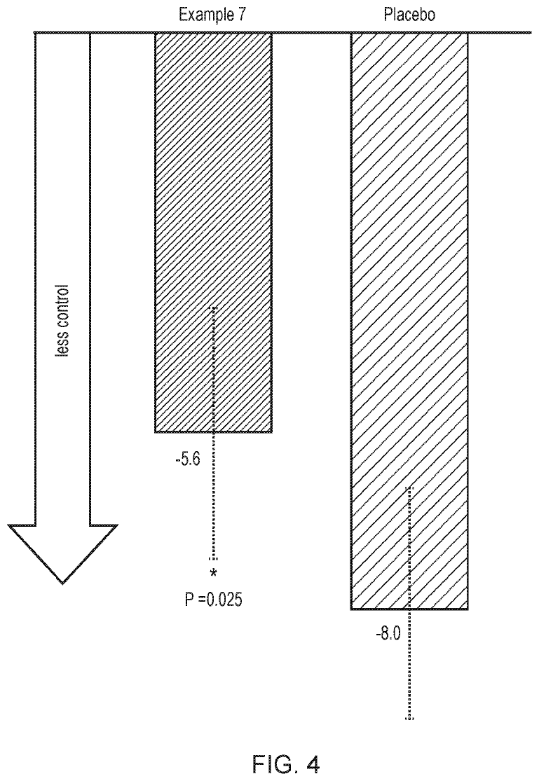

Feeling ‘in control’—Perceptions of being ‘in control’, measured by via a visual analogue scale (VAS), showed that participants perceived themselves to be more ‘in control’ compared with the placebo. The results are shown in FIGS. 2 and 4. FIG. 4 shows the overall result for all post-consumption time-points shown in FIG. 2 combined, for both the placebo and the oral product of Example 7.

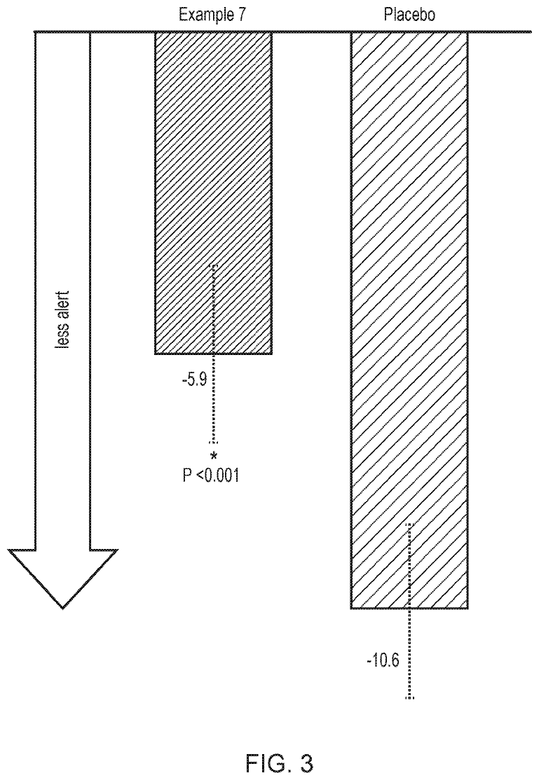

Alertness—Perceived alertness, measured by via a visual analogue mood scale VAMS, showed significantly more alertness compared with the placebo. This reduced alertness reduction was statistically different to the placebo at each time point and over the time course of the study. The results are shown in FIGS. 1 and 3. FIG. 3 shows the overall result for all post-consumption time-points shown in FIG. 1, for both the placebo and the oral product of Example 7.

Significant improvements for main effects versus placebo were observed for multiple endpoints. Interestingly, higher alertness and lower sleepiness were observed versus placebo demonstrating that the liquid dosage form of the present invention imparts relaxing effects, mood maintenance and stress management but without sedation/drowsiness.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

Further embodiments are defined in the following numbered clauses:

1. A liquid oral product comprising a combination of active ingredients, wherein the combination of active ingredients comprises:
(i) L-theanine;
(ii) ginseng;
(iii) vitamin C;
(iv) Echinacea; and optionally (v) vitamin D.
   2. A liquid oral product according to clause 1, wherein the L-theanine is present in an amount of from about 0.01% to about 15% by weight of the oral product.
   3. A liquid oral product according to clause 1 or 2, wherein the ginseng is present in an amount of from about 0.01% to about 5% by weight of the oral product.
   4. A liquid oral product according to any one of clauses 1 to 3, wherein the vitamin C is present in an amount of from about 0.1% to about 10% by weight of the oral product, and optionally when the vitamin D is present, vitamin D in an amount of from about 0.000001% to about 0.01% by weight of the oral product.
   5. A liquid oral product according to any one of clauses 1 to 4, wherein the Echinacea, is present in an amount of from about 0.001 wt % to about 20 wt % of the oral product.
   6. A liquid oral product according to any one of clauses 1 to 5, wherein the vitamin C and L-theanine are present in a weight ratio of from about 1:10 to about 10:1.
   7. A liquid oral product according to any one of clauses 1 to 6, wherein the L-theanine and ginseng are present in a weight ratio of from about 1:10 to about 35:1.
   8. A liquid oral product according to any one of clauses 1 to 7, wherein the Echinacea and L-theanine are present in a weight ratio of about 1:10 to about 25:1.
   9. A liquid oral product according to any one of clauses 1 to 9, wherein the combination of active ingredients further comprises lemon balm, optionally present in an amount of from about 0.01% to about 3% by weight of the oral product.
   10. A liquid oral product according to clause 9, wherein the L-theanine and lemon balm are present in a weight ratio of from about 1:10 to about 35:1.
   11. A liquid oral product according to any one of clauses 1 to 10, wherein the ginseng and lemon balm are present in a weight ratio of from about 1:10 to about 10:1.
   12. A liquid oral product according to any one of clauses 1 to 11, wherein the combination of active ingredients further comprises chamomile extract, optionally in an amount of from about 0.001% to about 1% by weight of the oral product.
   13. A liquid oral product according to clause 12, wherein the L-theanine and chamomile extract are present in a weight ratio of from about 1:1 to about 100:1.
   14. A liquid oral product according to clause 12 or clause 13, wherein the ginseng and chamomile extract are present in a weight ratio of from about 1:1 to about 25:1.
   15. A liquid oral product according to any one of clauses 1 to 14, further comprising one or more additives selected from the group consisting of a flavouring agent, sweetener, acidifying agent, preservative, and mixtures thereof.
   16. A liquid oral product according to any one of clauses 1 to 15, wherein the oral product further comprises zinc or zinc gluconate, optionally wherein the zinc or zinc gluconate is present in an amount of from about 0.001% to about 2% by weight of the oral product.
   17. A liquid oral product according to any one of clauses 1 to 16, wherein the liquid oral product has a volume of from about 1 mL to about 200 mL of liquid, preferably a volume of from about 20 mL to about 100 ml of liquid.
   18. A liquid oral product according to any one of clauses 1 to 17, wherein the oral product comprises the combination of active ingredients in an amount of from about 0.1% to about 15% by weight of the oral product.
   19. A liquid oral product according to any one of clauses 1 to 18, wherein the oral product comprises water in an amount of from about 50% to about 99.9% by weight of the oral product.
   20. A liquid oral product according to any one of clauses 1 to 19, wherein the combination of active ingredients comprises:
(i) L-theanine;
(ii) ginseng;
(iii) vitamin C;
(iv) Echinacea;
(v) vitamin D; and
(vi) optionally chamomile extract and/or
(vii) lemon balm.
   21. A liquid oral product according to any one of clauses 1 to 20, wherein the combination of active ingredients comprises:
(i) from about 25 mg to about 2000 mg of L-theanine;
(ii) from about 10 mg to about 500 mg of ginseng;
(iii) from about 50 mg to about 500 mg of vitamin C;
(iv) from about 10 mg to about 1500 mg of Echinacea;
(v) optionally from about 0.001 mg to about 1 mg of vitamin D; and
optionally (vi) from about 1 mg to about 100 mg of chamomile extract and/or
(vii) from about 1 mg to about 200 mg of lemon balm extract.
   22. A process for preparing a liquid oral product as defined in any one of clauses 1 to 21, the process comprising:
(a) combining active ingredients, wherein the active ingredients include: (i) L-theanine; (ii) ginseng; (iii) vitamin C; (iv) Echinacea; and optionally (v) vitamin D;
(b) contacting the active ingredients with water; and
(c) mixing the active ingredients with the water to prepare the liquid oral product.
   23. Use of a combination of active ingredients to boost immune response or to support immunity of a human or animal, wherein the combination of active ingredients comprises (i) L-theanine; (ii) ginseng; (iii) vitamin C; (iv) Echinacea; and optionally (v) vitamin D, preferably wherein the combination of active ingredients are present in a liquid oral product according to any one of clauses 1 to 21.
   24. Use of a combination of active ingredients to support resilience of a human or animal against infection, wherein the combination of active ingredients comprises (i) L-theanine; (ii) ginseng; (iii) vitamin C; (iv) Echinacea; and optionally (v) vitamin D, preferably wherein the combination of active ingredients are present in a liquid oral product according to any one of clauses 1 to 21.

1. A liquid oral product comprising a combination of active ingredients, wherein the combination of active ingredients comprises:
(i) L-theanine;
(ii) Ginseng;
(iii) vitamin C;
(iv) Echinacea;
v) lemon balm;
(vi) chamomile extract; and
optionally (vii) vitamin D or selenium.

2. A liquid oral product according to claim 1, wherein the L-theanine is present in an amount of from about 0.01% to about 15% by weight of the oral product.

3. A liquid oral product according to claim 1, wherein the Ginseng is present in an amount of from about 0.01% to about 5% by weight of the oral product.

4. A liquid oral product according to claim 1, wherein the vitamin C is present in an amount of from about 0.01% to about 10% by weight of the oral product, and optionally when the vitamin D is present, vitamin D in an amount of from about 0.000001% to about 0.1% by weight of the oral product, or optionally when the selenium is present, selenium in an amount of from about 0.000001% to about 0.001% by weight of the oral product.

5. (canceled)

6. A liquid oral product according to claim 1, wherein the Echinacea, is present in an amount of from about 0.001 wt % to about 20 wt % of the oral product.

7. A liquid oral product according to claim 1, wherein the vitamin C and L-theanine are present in a weight ratio of from about 1:10 to about 10:1.

8. A liquid oral product according to claim 1, wherein the L-theanine and Ginseng are present in a weight ratio of from about 1:10 to about 35:1.

9. A liquid oral product according to claim 1, wherein the Echinacea and L-theanine are present in a weight ratio of about 1:10 to about 25:1.

10. A liquid oral product according to claim 1, wherein the lemon balm is present in an amount of from about 0.01% to about 3% by weight of the oral product, optionally wherein the L-theanine and lemon balm are present in a weight ratio of from about 1:10 to about 35:1.

11. (canceled)

12. A liquid oral product according to claim 1, wherein the ginseng and lemon balm are present in a weight ratio of from about 1:10 to about 10:1.

13. A liquid oral product according to claim 1, wherein the chamomile extract is present in an amount of from about 0.001% to about 1% by weight of the oral product, optionally wherein the L-theanine and chamomile extract are present in a weight ratio of from about 1:1 to about 100:1.

14. (canceled)

15. (canceled)

16. (canceled)

17. A liquid oral product according to claim 1, wherein the oral product further comprises zinc or zinc gluconate, optionally wherein the zinc or zinc gluconate is present in an amount of from about 0.001% to about 2% by weight of the oral product.

18. A liquid oral product according to claim 1, wherein the liquid oral product has a volume of from about 1 mL to about 200 mL of liquid, optionally a volume of from about 20 mL to about 100 mL of liquid.

19. A liquid oral product according to claim 1, wherein the oral product comprises the combination of active ingredients in an amount of from about 0.1% to about 15% by weight of the oral product.

20. A liquid oral product according to claim 1, wherein the oral product comprises water in an amount of from about 50% to about 99.9% by weight of the oral product.

21. (canceled)

22. A liquid oral product according to claim 1, wherein the combination of active ingredients comprises:
(i) from about 25 mg to about 2000 mg of L-theanine;
(ii) from about 10 mg to about 500 mg of ginseng;
(iii) from about 10 mg to about 500 mg of vitamin C;
(iv) from about 10 mg to about 1500 mg of Echinacea;
(v)
from about 1 mg to about 100 mg of chamomile extract;
(vi) from about 1 mg to about 200 mg of lemon balm extract; and
(vii) optionally from about 0.001 mg to about 50 mg of vitamin D or from about 0.001 mg to about 0.2 mg of selenium.

23. A process for preparing a liquid oral product as defined in claim 1, the process comprising:
(a) combining the active ingredients;
(b) contacting the active ingredients with water; and
(c) mixing the active ingredients with the water to prepare the liquid oral product.

24. A method of boosting immune response or supporting immunity of a human or animal or supporting resilience of a human or animal against infection, wherein the method comprises supplying a combination of active ingredients to the human or animal, wherein the combination of active ingredients comprises (i) L-theanine; (ii) ginseng; (iii) vitamin C; (iv) Echinacea; (v) lemon balm; (vi) chamomile extract; and optionally (vii) vitamin D or selenium, optionally wherein the combination of active ingredients are present in a liquid oral product according to claim 1.

25. (canceled)

26. A method of improving alertness of a human or animal, wherein the method comprises supplying a combination of active ingredients to the human or animal, wherein the combination of active ingredients comprises (i) L-theanine; (ii) ginseng; (iii) vitamin C; (iv) Echinacea; (v) lemon balm; (vi) chamomile extract; and optionally (vii) vitamin D or selenium, optionally wherein the combination of active ingredients are present in a liquid oral product according to claim 1.

27. A method of improving feelings of being in control of a human or animal, wherein the method comprises supplying a combination of active ingredients to the human or animal, wherein the combination of active ingredients comprises (i) L-theanine; (ii) ginseng; (iii) vitamin C; (iv) Echinacea; (v) lemon balm; (vi) chamomile extract; and optionally (vii) vitamin D or selenium, optionally wherein the combination of active ingredients are present in a liquid oral product according to claim 1.

28. A method of reducing sleepiness of a human or animal, wherein the method comprises supplying a combination of active ingredients to the human or animal, wherein the combination of active ingredients comprises (i) L-theanine; (ii) ginseng; (iii) vitamin C; (iv) Echinacea; (v) lemon balm; (vi) chamomile extract; and optionally (vii) vitamin D or selenium, optionally wherein the combination of active ingredients are present in a liquid oral product according to claim 1.
